SEQUENCE LISTING (32 sequences)

SEQ ID NO: 1 - protein, 459 aa, Artificial Sequence
  synthetic sequence
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 2 - protein, 219 aa, Artificial Sequence
  synthetic sequence
ESGYAQNGDLEDAELDDYSFSCYSQLEVNGSQHSLTCAFEDPDVNTTNLEFEICGALVEVKCLNFRKLQEIYFIETKKFLLIGKSNICVKVGEKSLTCKKIDLTTIVKPEAPFDLSVIYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 3 - protein, 9 aa, Artificial Sequence
  synthetic sequence
FTLSDYYMA

SEQ ID NO: 4 - protein, 17 aa, Artificial Sequence
  synthetic sequence
TISASGLRTYYPDSVKG

SEQ ID NO: 5 - protein, 13 aa, Artificial Sequence
  synthetic sequence
PMSAHYGFNYFDY

SEQ ID NO: 6 - protein, 13 aa, Artificial Sequence
  synthetic sequence
PLSAHYGFNYFDY

SEQ ID NO: 7 - protein, 11 aa, Artificial Sequence
  synthetic sequence
RTSEDIYNGLA

SEQ ID NO: 8 - protein, 12 aa, Artificial Sequence
  synthetic sequence
CRTSEDIYQGLA

SEQ ID NO: 9 - protein, 7 aa, Artificial Sequence
  synthetic sequence
SANSLHI

SEQ ID NO: 10 - protein, 7 aa, Artificial Sequence
  synthetic sequence
SANTLHI

SEQ ID NO: 11 - protein, 9 aa, Artificial Sequence
  synthetic sequence
QQYYDYPLA

SEQ ID NO: 12 - protein, 122 aa, Artificial Sequence
  synthetic sequence
AVQLVESGGGLVQPGGSLKLSCAVSGFTLSDYYMAWVRQAPKKGLEWVATISASGLRTYYPDSVKGRFTISRDDAKRSLFLQMTSLKSEDTATYYCARPMSAHYGFNYFDYWGQGVMVTVSS

SEQ ID NO: 13 - protein, 122 aa, Artificial Sequence
  synthetic sequence
QVQLVESGGGLVKPGGSLRLSCAVSGFTLSDYYMAWVRQAPGKGLEWVATISASGLRTYYPDSVKGRFTISRDDAKNSLYLQMNSLRAEDTAVYYCARPMSAHYGFNYFDYWGQGTLVTVSS

SEQ ID NO: 14 - protein, 122 aa, Artificial Sequence
  synthetic sequence
QVQLVESGGGLVKPGGSLRLSCAVSGFTLSDYYMAWIRQAPGKGLEWVSTISASGLRTYYPDSVKGRFTISRDNAKNSLYLQMNSLRAEDTAVYYCARPMSAHYGFNYFDYWGQGTLVTVSS

SEQ ID NO: 15 - protein, 122 aa, Artificial Sequence
  synthetic sequence
QVQLVESGGGLVKPGGSLRLSCAVSGFTLSDYYMAWIRQAPGKGLEWVSTISASGLRTYYPDSVKGRFTISRDNAKNSLYLQMNSLRAEDTAVYYCARPLSAHYGFNYFDYWGQGTLVTVSS

SEQ ID NO: 16 - protein, 108 aa, Artificial Sequence
  synthetic sequence
DIQMTQSPASLSASLGETVTIECRTSEDIYNGLAWYQQKPGKSPQLLVYSANSLHIGVPSRFSGSGSGTQYSLKINSLQFEDVASYFCQQYYDYPLAFGSGTKLEIKR

SEQ ID NO: 17 - protein, 107 aa, Artificial Sequence
  synthetic sequence
EIVMTQSPATLSVSPGERATLSCRTSEDIYNGLAWYQQKPGQAPRLLVYSANSLHIGIPARFSGSGSGTEYTLTISSLQSEDFAVYFCQQYYDYPLAFGGGTKVEIK

SEQ ID NO: 18 - protein, 107 aa, Artificial Sequence
  synthetic sequence
EIVMTQSPATLSVSPGERATLSCRTSEDIYNGLAWYQQKPGQAPRLLIYSANSLHIGIPARFSGSGSGTEFTLTISSLQSEDFAVYYCQQYYDYPLAFGGGTKVEIK

SEQ ID NO: 19 - protein, 107 aa, Artificial Sequence
  synthetic sequence
EIVMTQSPATLSVSPGERATLSCRTSEDIYQGLAWYQQKPGQAPRLLIYSANTLHIGIPARFSGSGSGTEFTLTISSLQSEDFAVYYCQQYYDYPLAFGGGTKVEIK

SEQ ID NO: 20 - protein, 449 aa, Artificial Sequence
  synthetic sequence
QVQLVESGGGLVKPGGSLRLSCAVSGFTLSDYYMAWIRQAPGKGLEWVSTISASGLRTYYPDSVKGRFTISRDNAKNSLYLQMNSLRAEDTAVYYCARPLSAHYGFNYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 21 - protein, 213 aa, Artificial Sequence
  synthetic sequence
EIVMTQSPATLSVSPGERATLSCRTSEDIYQGLAWYQQKPGQAPRLLIYSANTLHIGIPARFSGSGSGTEFTLTISSLQSEDFAVYYCQQYYDYPLAFGGGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 122 aa, Artificial Sequence; identical to SEQ ID NO: 15
  synthetic sequence

SEQ ID NO: 23 - protein, 107 aa, Artificial Sequence
  synthetic sequence
DIQMTQSPSSLSASVGDRVTITCRTSEDIYQGLAWYQQKPGKAPKLLVYSANTLHIGVPSRFSGSGSGTDYTLTISSLQPEDFATYFCQQYYDYPLAFGGGTKVEIK

SEQ ID NO: 24 - protein, 107 aa, Artificial Sequence
  synthetic sequence
DIQMTQSPSSLSASVGDRVTITCRTSEDIYQGLAWYQQKPGKAPKLLLYSANTLHIGVPSRFSGSGSGTDYTLTISSLQPEDFATYFCQQYYDYPLAFGGGTKVEIK

SEQ ID NO: 25 - protein, 107 aa, Artificial Sequence
  synthetic sequence
DIQMTQSPSSLSASVGDRVTITCRTSEDIYQGLAWYQQKPGKAPKLLVYSANTLHIGVPSRFSGSGSGTDYTLTISSLQPEDFATYYCQQYYDYPLAFGGGTKVEIK

SEQ ID NO: 26 - protein, 107 aa, Artificial Sequence
  synthetic sequence
DIQMTQSPSSLSASVGDRVTITCRTSEDIYQGLAWYQQKPGKAPKLLLYSANTLHIGVPSRFSGSGSGTDYTLTISSLQPEDFATYYCQQYYDYPLAFGGGTKVEIK

SEQ ID NO: 27 - protein, 449 aa, Artificial Sequence; identical to SEQ ID NO: 20
  synthetic sequence

SEQ ID NO: 28 - protein, 214 aa, Artificial Sequence
  synthetic sequence
DIQMTQSPSSLSASVGDRVTITCRTSEDIYQGLAWYQQKPGKAPKLLLYSANTLHIGVPSRFSGSGSGTDYTLTISSLQPEDFATYYCQQYYDYPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 213 aa, Artificial Sequence
  synthetic sequence
DIQMTQSPSSLSASVGDRVTITCRTSEDIYQGLAWYQQKPGKAPKLLLYSANTLHIGVPSRFSGSGSGTDYTLTISSLQPEDFATYYCQQYYDYPLAFGGGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 327 aa, Artificial Sequence
  synthetic sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 31 - protein, 107 aa, Artificial Sequence [sequence omitted]

SEQ ID NO: 32 - protein, 106 aa, Artificial Sequence
  synthetic sequence
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0389104 A1
(43) Pub. Date: Dec. 8, 2022

(54) Method for Treating CD127-Positive Cancers by Administering an Anti-CD127 Agent

(71) Applicant: OSE IMMUNOTHERAPEUTICS, Nantes (FR)

(72) Inventors: Nicolas POIRIER, Grandchamps des Fontaines (FR); Irène BACCELLI, NANTES (FR); Caroline MARY, SAINTE-PAZANNE (FR); Sabrina PENGAM, ST LUCE S/ LOIRE (FR)

(21) Appl. No.: 17/334,158

(22) Filed: May 28, 2021

(51) Int. Cl. C07K 16/28; A61P 35/02; A61K 31/573

(52) U.S. Cl. CPC C07K 16/2866; A61P 35/02; A61K 31/573

(57) ABSTRACT

The invention pertains to the field of immunotherapy. The present invention provides a new use of anti-CD127 agent, in particular anti-CD127 antibodies or related compounds for the treatment and/or the prevention of cancer.

The invention relates to a method for treating a patient having a CD127-positive cancer, in particular a CD127-positive leukemia, by administering to the patient a therapeutic dose of an anti-CD127 agent, the anti-CD127 agent having the capability to enhance the Antibody Dependent Cellular Phagocytosis (ADCP) activity of macrophages targeting CD127-positive cancer cells, and that does not have Antibody Dependent Cytotoxic Activity (ADCC), in particular on immune cells, more particularly on T cells.

### REFERENCE TO SEQUENCE LISTING SUBMITTED ELECTRONICALLY

The content of the electronically submitted sequence listing in ASCII text file (Name TC_B14422_ST25.txt; Size: 40838 bytes; and Date of Creation: May 28, 2021) filed with the application is incorporated herein by reference in its entirety.

### FIELD OF THE INVENTION

The invention pertains to the field of immunotherapy. The present invention provides a new use of anti-CD127 agent, in particular anti-CD127 antibodies or related compounds for the treatment and/or the prevention of cancer.

The invention relates to an anti-CD127 agent for use in the treatment of a patient having a CD127-positive cancer and a method for treating a patient having a CD127-positive cancer, in particular a CD127-positive leukemia, by administering to the patient a therapeutic dose of an anti-CD127 agent, the anti-CD127 agent having Antibody Dependent Cellular Phagocytosis (ADCP) activity on CD127-positive tumor cells, in particular by macrophages cells, and that does not have Antibody Dependent Cytotoxic Activity (ADCC), in particular on immune cells, more particularly on T cells.

The present invention also relates to a method for promoting phagocytosis of CD127-positive cells, in particular CD127-positive tumor cells, in particular by macrophages, by administering to a patient in need thereof a therapeutic amount of an anti-CD127 agent that has Antibody Dependent Cellular Phagocytosis (ADCP) activity on CD127-positive tumor cells, in particular by macrophages cells, and that does not have Antibody Dependent Cytotoxic Activity (ADCC), in particular on immune cells, more particularly on T cells.

The invention also provides an anti-CD127 agent that has Antibody Dependent Cellular Phagocytosis (ADCP) activity on CD127-positive tumor cells, in particular by macrophages cells, and that does not have Antibody Dependent Cytotoxic Activity (ADCC), in particular on immune cells, more particularly on T cells, for the treatment of a patient having CD127-positive tumor cells, in particular CD127-positive leukemia, more particularly having CD127-positive Acute Lymphoblastic Leukemia.

The invention also relates to an anti-CD127 agent for use in the treatment of a patient having a CD127-positive cancer and a method for treating a patient having a CD127-positive cancer, in particular a CD127-positive leukemia, by administering to the patient a therapeutic dose of an anti-CD127 agent, the anti-CD127 agent having Antibody Dependent Cellular Phagocytosis (ADCP) activity on CD127-positive tumor cells, in particular by macrophages cells.

The invention also provides an anti-CD127 agent which enhances Antibody Dependent Cellular Phagocytosis (ADCP) activity of macrophages targeting CD127-positive cancer cells, for the treatment of a patient having CD127-positive tumor cells, in particular CD127-positive leukemia, more particularly having CD127-positive Acute Lymphoblastic Leukemia.

### BACKGROUND OF THE INVENTION

Cancer is a major worldwide health concern causing the death of approximately 9.5 million people a year, while more than 20 million people develop a cancer within a year (world cancer report by World Health Organization, 2018). Several malignant cells have been shown to display CD127 expression. This is for example the case for Sezary cutaneous lymphoma (60% of them), or childhood acute lymphoblastic leukemia in which about 15% of the children develop gain-of-function mutation in CD127, rendering these tumors partially IL-7 dependent (Shochat et al., 2011). Acute lymphoblastic leukemia (ALL) is a cancer of the lymphoid line of blood cells characterized by the development of large numbers of immature lymphocytes. ALL progresses rapidly and is typically fatal within weeks or months if left untreated. The underlying mechanism involves multiple genetic mutations that results in rapid cell division. The excessive immature lymphocytes in the bone marrow interfere with the production of new red blood cells, white blood cells, and platelets.

ALL is typically treated initially with chemotherapy. This is then followed by further chemotherapy typically over a number of years. Chemotherapy treatments usually cause several side effects including but not limited to fatigue, hair loss, easy bruising and bleeding, infection, anemia (low red blood cell counts), nausea and vomiting. Additional and/or different treatments of ALL may include intrathecal chemotherapy or radiation therapy. Stem cell transplantation may be used if the disease recurs following standard treatment.

Despite numerous existing treatments, there is still a need for an improved method of treating cancer. New treatments based on antibody technology such as chimeric antigen receptor T cell (CAR-T cell) immunotherapy and use of monoclonal antibodies are being used and further studied. Indeed, several strategies involving the administration of CAR-T cells, anti-CD3 antibodies and anti-CD19 antibodies are currently in development for treating patients developing an ALL. Nonetheless, these strategies have drawbacks, among which one can cite the cost of these therapies and mostly the toxicity associated with the administered compounds. Due to the ubiquitous expression of the target CD19 marker in all B-lineage subtypes, these strategies may lead to collateral damages within the host during the treatment of ALL. Accordingly, there is a need for new treatments of ALL, which do not have the same drawbacks as the treatment of the prior art.

CD127 is part of the heterodimeric IL-7 receptor that is composed of CD127 and the common γ chain, which is shared by other cytokine receptors (IL-2R, IL-4R, IL-9R, IL-15R, and IL-21R). CD127 is expressed on thymocytes, T- and B-cell progenitors, mature T cells, monocytes, and some other lymphoid and myeloid cells. Studies have shown that IL-7R plays an important role in the proliferation and differentiation of mature T cells. Further, signaling induced by the dimerization of CD127 with the common γ chain plays a pivotal role in T-cell development and maintenance of T-cell memory. Expression of CD127 is commonly associated with central and effector memory functions in both CD4 and CD8 peripheral T cells.

The inventors have identified that anti-CD127 agents that have Antibody Dependent Cellular Phagocytosis (ADCP) activity on CD127-positive tumor cells, in particular by macrophages cells, and that do not have Antibody Dependent Cytotoxic Activity (ADCC), in particular on immune cells, more particularly on T cells, may be useful in the treatment of CD-127 positive cancers, in particular in the treatment of CD127-positive ALL. By administering an anti-CD127 agent as defined, the inventors observed that phagocytosis of cancer cells by immune cells of the host is increased. Thus, the administration of an anti-CD127 agent that has Antibody Dependent Cellular Phagocytosis (ADCP) activity on CD127-positive tumor cells, in particular by macrophages cells, and that does not have Antibody Dependent Cytotoxic Activity (ADCC), in particular on immune cells, more particularly on T cells, to a patient in need thereof leads to the phagocytosis of cancer cells expressing CD127. The narrower cell profile of molecular expression of CD127 as compared to the more ubiquitous expression of CD19 in the B lineage is clearly an asset in the treatment of patients having a cancer, in particular in the treatment of patients having a CD127-positive cancer, more particularly in patients having an ALL, by lowering potential side effects of treatment targeting more ubiquitous molecules, especially receptors, as compared to the expression of CD127.

### SUMMARY OF THE INVENTION

The present inventors showed for the first time that an anti-CD127 agent as defined herein that has the capability to increase the Antibody Dependent Cellular Phagocytosis (ADCP) activity of macrophages targeting CD127-positive cancer cells but that does not have Antibody Dependent Cytotoxic Activity (ADCC) on CD127-positive tumor cells enhances the phagocytosis of tumor cells and improves the survival rate of leukemia preclinical mouse models. More particularly, the administration of an anti-CD127 agent as defined herein increases the phagocytosis of ALL tumor cells, more particularly of T-cell ALL tumor cells and/or B-cell ALL tumor cells. More particularly, the inventors illustrated that anti-CD127 antibodies having an IgG4 immunoglobulin domain have the capability to increase the ADCP of CD127-positive tumor cells, while ADCP of normal cells, in particular of normal T cells, is not significantly impacted as compared to the increase in the phagocytosis of tumor cells .

Antibodies kill targeted tumor cells by several mechanisms, including antibody-dependent cell-mediated cytotoxicity (ADCC) and Antibody Dependent Cellular Phagocytosis (ADCP). In antibody-dependent cellular cytotoxicity (ADCC), an Fcγ receptor (FcγR or FCGR) on the surface of an immune effector cell binds to the Fc region of an antibody, which specifically binds to a target cell. Cells that can mediate ADCC are non-specific cytotoxic cells. In contrast, Antibody-dependent cellular phagocytosis (ADCP) relies on phagocytes to devour target cells. ADCP is a highly regulated process in which an antibody eliminates binding target and initiates phagocytosis by linking its Fc domain to a specific Fcγ receptor on the phagocytic cell. Unlike ADCC, ADCP can be mediated by monocytes, macrophages, neutrophils and dendritic cells via FcγRIIa (CD32a), FcγRI (CD64) and FcγRIIIa (CD16), where FcγRIIa (CD32a) on macrophages represents the major pathway. These three CD markers are able to interact with the Fc portion of antibodies and thus bind to antibodies, thereby inducing ADCC and/or ADCP.

Anti-CD127 agents, in particular anti-CD127 antibodies, more particularly anti-CD127 antagonist antibodies or related compounds, are known to be useful in the treatment of autoimmune diseases and inflammatory diseases by directly interacting with the cells of the immune system of the host. In particular, these anti-CD127 agents are known to be useful in the treatment of autoimmune diseases and inflammatory diseases by inhibiting the survival of antigen-engaged memory T cell survival, without impact on quiescent human T cells (Belarif et al. Nature Communication 2018). Nonetheless, the use of anti-CD127 agents having an ADCP capability but not ADCC capability against CD127-positive tumor cells for treating CD127-positive cancers by enhancing and/or inducing the phagocytosis of CD127-positive tumor cells by immune cells of the host, in particular by macrophages, was not known or suggested. Further, due to the specific expression of CD127 in the lymphoid cell lineage, it is interestingly observed that side effects associated with the administration of an anti-CD127 agent as defined here above (i.e. having an ADCP capability but no ADCC capability against CD127-positive tumor cells) to treat a CD127-positive cancer are limited as compared to treatments currently in development, which target molecules with either an ubiquitous expression or a broad expression in different cell lineages (like CD19). In particular, the inventors illustrate for the first time that anti-CD127 antibodies are able on their own to enhance or initiate the phagocytosis of CD127-positive tumor cells by immune cells of the host. It is illustrated for the first time that anti-CD127 antibodies, in particular anti-CD127-IgG4 antibodies, are able to increase on their own the phagocytosis of CD127-positive tumor cells by the ADCP mechanism, without the need of other therapeutic agents.

To sum up, the present invention of using an anti-CD127 agent that has an ADCP capability but no ADCC capability against CD127-positive tumor cells or the method comprising the administration of such an agent to treat a patient having CD127-positive tumor cells has the following advantages:
it is a targeted cancer therapy with a better focus since CD127 is expressed in less cell lineages than other targets of targeted cancer therapies, like CD19;
it lacks cytotoxicity against immune cells of the patient who is treated with such an agent, in particular it does not reduce the number of macrophages and/or T cells of the patient;
it enhances the survival rate and the survival length.

It was not obvious to treat patients having a cancer, in particular leukemia, and more particularly ALL, with tumor cells expressing CD127 with an anti-CD127 agent that has an ADCP capability but no ADCC capability against CD127-positive tumor cells. As an example, tumor cells depend on IL7R signaling for their survival, but the survival of these cells is not linked to the level of expression of CD127. IL7R targeting with monoclonal antibodies in ALL has been investigated mainly based on the observation that T-ALL and B-ALL cells, including those with leukemic stem cell activities, are dependent on the activity of the IL7R pathway, itself mediating a pro-survival and pro-proliferation signaling cascade (Gonzalez-Garcia et al., Blood 2019, Alsadeq A., et al. Blood 2018, Abdelrasoul et al., Nature Communication 2020, Akkapeddi et al. Leukemia 2019). This dependency on the IL7R pathway has been molecularly dissected in the case of Philadelphia chromosome positive leukemias, where the IL7R was found to interact with CXCR4 and to recruit BCR-ABL1 and JAK kinases to mediate the leukemogenic effect of the BCR-ABL1 oncogene (Abdelrasoul et al., Nature Communication 2020). The dependency of ALL cells to the IL7R signaling cascade is therefore not directly linked to the level of expression of CD127, as Philadelphia-chromosome positive leukemias generally have low baseline expression levels of the receptor (ProteinPaint Study, St Jude Hospital).

The inventors surprisingly observed that anti-CD127 agent that has an ADCP capability but no ADCC capability against CD127-positive tumor cells, like the antibody referenced N13B2-hVL6 in the working examples of the invention, had an antileukemic activity. N13B2-hVL6 is an antagonist monoclonal antibody directed against CD127 (Belarif et al., Nature Communication 2018) and as such was hypothesized to mediate its antileukemic effect primarily due to its capacity to efficiently block the pro-survival and pro-proliferation signaling cascade mediated by the IL7-IL7R pathway. Surprisingly, the inventors found that the antileukemic effect of N13B2-hVL6 was at least partly mediated by its capacity to induce ADCP, a mechanism which has not yet been reported for anti-IL7R antibodies for the treatment of ALL. Due to this robust ADCP induction, leukemias with high CD127 expression, regardless of their functional dependency on the IL7R pathway are predicted to positively respond to a treatment with an anti-CD127 agent as defined here above, thereby opening therapeutic opportunities for any patient with sufficient expression of CD127 at the surface of their tumor cells, in particular when these tumor cells are associated to leukemia, more particularly to ALL. Of importance, this includes IL7R mutated ALLs with constitutive activation of the pathway, which are highly prevalent in T-ALL, such as the DND41 cell line. It was previously predicted to be refractory to IL7R targeting by N13B2-hVL6 since constitutive activation mutations of IL7R cannot be antagonized by this type of antibodies.

Further, the inventors surprisingly found that the anti-CD127 agents lacking ADCC capabilities had strong ADCP capabilitie and are effective on CD127 positive tumor cells. ADCC and ADCP both rely on the binding to FcγRest. For example, N13B2-hVL6 is an IgG4 antibody which lacks ADCC capacities, as do most IgG4 antibodies, since IgG4 formats have weak affinities for all FC receptors except FcγRI and are therefore poor inducers of Fc-mediated effector functions (Yu et al. Journal of Hematology & Oncology, Tay et al. Front Immunol 2019). An antibody that lacks ADCC capacity is expected to be unable to induce other Fc-mediated effector functions such as ADCP, as all Fc-mediated effector mechanisms rely on the binding of the Fc portion of antibodies to Fc gamma receptors. As such, providing anti-CD127 agents with the capacity to induce high levels of ADCP without inducing a high level of ADCC is highly unexpected.

The inventors were also very surprised to find that anti-CD127 IgG4 antibodies, N13B2-hVL6 for example, has the capability to induce ADCP. IgG4 antibodies usually lack Fc-mediated effector functions such as ADCP. IgG4 format antibodies have weak affinities for all FC receptors except FcγRI and are therefore poor inducers of Fc-mediated effector functions (Yu et al. Journal of Hematology & Oncology, Tay et al. Front Immunol 2019). Illustratively, all currently approved anti-PD-1 antibodies are in the IgG4 format to avoid Fc-mediated elimination of PD1+ CD8+ T lymphocytes. Conversely, antibodies directed against CTLA-4 (for instance Ipilimumab), which is expressed by immunosuppressive Treg cells in the tumor microenvironment (TME), are used in the clinic in IgG1 format to promote elimination of Tregs via ADCC/ADCP mechanisms (Du et al., Cell Res 2018). Surprisingly, although N13B2-hVL6 is an IgG4 antibody, the inventors found that it was able to induce strong levels of ADCP that can be superior to that of reference pro-phagocytic anti-CD47 antibodies (for example in CD127-high leukemia cell lines such as REH). Also unexpectedly, N13B2-hVL6 displays superior ADCP capacities compared to all other IgG1 format anti-CD127 antibodies (1A11, HAL and Effie3-VH3VL3) in all tested leukemia cell lines.

Moreover, the inventors also illustrate that the anti-CD127 agent to be used in the method of the invention, or for use according to the invention, do not have or have a no significant effect on ADCP capability on normal T cells, as compared to other therapeutic compounds usually administered in the treatment of cancers, in particular leukemia, and more particularly T-cell and B-cell leukemia. Thus, the use of the anti-CD127 agent according to the method of the invention leads to an increase in or to initiate the phagocytosis of CD127-positive tumor cells, while it does not deplete the normal T cell population of the host, thereby reducing side effects usually associated with multiple antibody-related treatment of cancers. In particular, the inventors show for the first time that administering an anti-CD127 agent as defined herein to healthy humans does not lead to lymphodepletion. Further, no adverse effects were observed in these healthy volunteers, on the contrary to observation in humans receiving the drugs currently administrated which severe adverse effects, like anti CD3-agent or anti-CD19 agent) (see Condo-Royo et al., Management of adverse effects of new monoclonal antibody treatments in acute lymphoblastic leukemia, Drugs Context 2020 Oct. 14; 9:2020-7-2).

In one aspect, the present invention relates to an anti-CD127 agent for use in the treatment of CD127-positive cancer, particularly by phagocytosis of CD127-positive tumor cells, in particular by macrophages, in particular a CD127-positive ALL, more particularly a CD127-positive T-cell ALL or a B-cell ALL.

In another aspect, the present invention relates to an anti-CD127 agent for use in the treatment of a patient having a CD127-positive cancer, particularly by phagocytosis of CD127-positive tumor cells, in particular by macrophages, in particular a CD127-positive ALL, more particularly a CD127-positive T-cell ALL or a B-cell ALL.

In another aspect, the present invention relates to a method for treating a patient having a CD127-positive cancer, in particular a patient having a T-cell ALL or a B-cell ALL, by increasing the phagocytosis of CD127-positive tumor cells, in particular by macrophages of the patient.

The present invention also relates to the use of an anti-CD127 agent for the manufacture of a medicament for the treatment of cancer, by enhancing the phagocytosis of CD127-positive tumor cells.

In some embodiments, a therapeutically effective amount of the anti-CD127 agent for use in the method of the invention or for use according to the invention is administered to a subject having a cancer.

The present invention further relates to anti-CD127 agent having (i.e. increasing as compared to a negative control which can be an isotype control such as MOTA-hIgG4) Antibody Dependent Cellular Phagocytosis (ADCP) activity of macrophages targeting CD127-positive cancer cells, in particular wherein said activity is achieved by or involves macrophages, for the treatment of a patient having a CD127-positive cancer, in particular a patient having leukemia, more particularly having ALL.

The present invention also relates to anti-CD127 antibodies or antigen-binding fragments thereof, having (i.e. increasing as compared to a negative control) Antibody Dependent Cellular Phagocytosis (ADCP) activity of macrophages targeting CD127-positive cancer cells, in particular wherein said activity is achieved by or involves macrophages, for the treatment of a patient having an ALL selected from the group consisting of CD127 overexpressing ALL (an overexpressing ALL may be determined by comparing CD127 expression in ALL cells as compared to CD127 expression in healthy bone marrow), CD127 and/or JAK-STAT pathway mutated ALL (as opposed to healthy cells), including BCR-ABL1-like ALL, as well as B cell precursor ALL bearing the following cytogenetics: t(1;19), t(12;21), MLL-rearrangements, Hyperdiploid karyotypes, trisomy 4 and trisomy 10. In a particular embodiment, the invention relates to anti-CD127 antibodies or antigen-binding fragments thereof, having (i.e. increasing as compared to a negative control) Antibody Dependent Cellular Phagocytosis (ADCP) activity macrophages targeting CD127-positive cancer cells, in particular wherein said activity is achieved by or involves macrophages, for the treatment of ALL selected from the group consisting of CD127 wild type T-ALL (HPB-ALL cell line), CD127-mutated T-ALL (DND41 cell line), t(1;19) B-ALL (697 cell line), t(12;21) B-ALL (REH cell line) and t(5;12) B-ALL (NALM6 cell line).

In another aspect, the invention relates to a pharmaceutical composition, suitable for administration to a mammalian host, in particular a human host, comprising an anti-CD127 agent as defined herein, and a pharmaceutical vehicle.

In particular, the invention relates to the pharmaceutical composition for use in the treatment of a patient, in particular a human patient having a CD127-positive cancer, the composition comprising an anti-CD127 agent that increases the phagocytosis of CD127-positive tumor cells, and increasing (as compared to a negative control) Antibody Dependent Cellular Phagocytosis (ADCP) activity of macrophages targeting CD127-positive cancer cells, and a pharmaceutical vehicle.

The present invention also relates to a combination of therapeutic agents for treating a patient having a CD127 positive cancer, in particular in leukemia, more particularly ALL, said combination comprising an anti-CD127 agent which increases the phagocytosis of CD127-positive tumor cells, and having (i.e. increasing as compared to a negative control) Antibody Dependent Cellular Phagocytosis (ADCP) activity of macrophages targeting CD127-positive cancer cells, the combination further comprising at least a second (i.e. distinct) therapeutic agent.

In a particular embodiment, the invention relates to the use of anti-CD127 agents as defined herein in order to deplete subpopulations of CD127-positive tumor cells, in particular CD127-positive tumor T cells and/or CD127-positive tumor B-cells in a patient having a cancer, in particular a cancer selected in the group of cancers specified herein, by enhancing the phagocytosis of CD127-positive tumor cells, in particular by macrophages.

### DETAILED DESCRIPTION OF THE INVENTION

### Definitions

As used herein, “antibody” includes polyclonal, monoclonal, recombinant, chimeric, humanized, bispecific, multispecific and modified antibodies, as well as monovalent and divalent antigen-binding fragments thereof. Furthermore, “antibody” includes synthetic antibodies, single chain antibodies, and fragments thereof. The antibody may be a human or nonhuman antibody. A nonhuman antibody may be humanized by recombinant methods to reduce its immunogenicity in human. More specifically the term “antibody” refers to a monoclonal antibody or recombinant monoclonal antibodies, or an antigen-binding fragment thereof.

As used herein, a “monoclonal antibody” is intended to refer to a preparation of antibody molecules, wherein antibodies share a common heavy chain and common light chain amino acid sequence, in contrast with “polyclonal” antibody preparations which contain a mixture of antibodies of different amino acid sequences. Monoclonal antibodies can be generated by several known technologies like phage, bacteria, yeast or ribosomal display, as well as by classical methods exemplified by hybridoma-derived antibodies. Thus, the term “monoclonal” is used to refer to all antibodies derived from one nucleic acid clone.

As used herein, an “antigen-binding fragment of an antibody” means a part of an antibody, i.e. a molecule corresponding to a portion of the structure of the antibody of the invention, that exhibits antigen-binding capacity for CD127, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragments that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies. For illustration purpose of specific embodiments of the invention, antigen binding fragments of an antibody that contain the variable domains comprising the CDRs of said antibody encompass Fv, dsFv, scFv, Fab, Fab′, F(ab′)2.

Antibodies and antigen-binding fragments of antibodies comprise at least a light chain variable domain and a heavy chain variable domain, each one comprising three hypervariable domains designated CDRs (Complementary Determining Regions). These domains encompass the recognition site for the antigen, i.e. CD127, in particular human CD127, and most particularly the extracellular domain of human CD127, thereby defining antigen recognition specificity.

Each Light and Heavy chain variable domains (respectively VL and VH) has three CDRs, designated VL-CDR1 (or LCDR1), VL-CDR2 (or LCDR2), VL-CDR3 (or LCDR3) and VH-CDR1 (or HCDR1), VH-CDR2 (or HCDR2), VH-CDR3 (or HCDR3), respectively.

Antibodies and antigen-binding fragments thereof may comprise or derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

Antigen-binding antibody mimetics are organic compounds that specifically bind antigens, but that are not structurally related to antibodies. They are usually artificial peptides or small proteins with a molar mass of about 3 to 20 kDa. Nucleic acids and small molecules are sometimes considered antibody mimetics as well, but not artificial antibodies, antibody fragments and fusion proteins composed from these. Common advantages over antibodies are better solubility, tissue penetration, stability towards heat and enzymes, and comparatively low production costs. Antibody mimetics are being developed as therapeutic and diagnostic agents. Antigen-binding antibody mimetics may also be selected among the group comprising affibodies, affilins, affimers, affitins, DARPins, and Monobodies.

As used herein, the term “specifically binds to” or “binds specifically” refers to the capability of anti-CD127 agent to be used in the method of the invention or for use according to the invention to interact with CD127 and to bind with CD127, preferably human CD127, while they do not bind or they bind with a significantly weaker binding affinity to other molecules, in particular to other proteins. Binding and binding specificity can be assayed by SPR (Surface Plasmon Resonance e.g. Biacore), ELISA or Western Blot analysis. In a particular embodiment, the ability of the anti-CD127 agent to bind to CD127 is considered to be specific when the binding affinity is of at least about 1×10−6 M, 1×10−7 M, 1×10−8 M, 1×10−9 M, 1×10−10 M, 1×10−11 M, 1×10−12 M, or more, and/or bind to a target with an affinity that is at least two-fold greater than its affinity for a nonspecific protein.

As used herein, the term “CD127” relates to a CD127 from a mammal species, preferably a human CD127, and most preferably human CD127 of SEQ ID No. 1. CD127, also known as Interleukin-7 receptor subunit alpha (IL7R-α), is a protein that in humans is encoded by the IL7R gene. CD127 is a type I cytokine receptor and is a subunit of the functional Interleukin-7 receptor and Thymic Stromal Lymphopoietin (TSLP) receptors. CD127 may correspond to the protein referenced under NCBI Sequence No. NP_002176.2. Alternatively, CD127 may correspond to a protein having the amino acid sequence of SEQ ID No. 1. The extracellular domain of CD127, which is likely to be recognized and bound to by anti-CD127 agent used in the invention may correspond to the amino acid sequence of SEQ ID No. 2.

The terms “cancer” and “tumor” have their general meaning in the art and refers to a group of diseases involving abnormal cell growth with the potential to invade or spread to other parts of the body. The term “cancer” further encompasses both primary and metastatic cancers.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, enabling to decrease the administered dose of one or more other medications required or used to treat the disease, increasing the quality of life, and/or prolonging survival, preventing or alleviating side-effects of chemotherapeutic treatment, or novel treatments of ALL, such as the severe cytokine release syndrome and neurotoxic side effects of the treatments comprising administration of anti-CD3 anti-CD19 bi-specific antibodies.

As used herein the term “antibody-dependent cell-mediated phagocytosis” or ‘ADCP”, refers to a cell-mediated reaction in which phagocytes, in particular macrophages, recognize bound antibody on a target cell and subsequently engulf the target cells, leading to their digestion within phagosomes. An anti-CD127 agent may be considered to have an ADCP capacity on CD127-positive tumor cells, in particular by macrophages cells, when it is able to induce a phagocytic score of strictly over 4.5. The phagocytic score is the fold change of percentage of CPD/CTG double positive cells within CTG positive cells (macrophages) as compared to a relevant isotype control, multiplied by the fold change of geometric mean of CPD fluorescence within CTG positive cells (macrophages) as compared to a relevant isotype control.

As used herein the term “Antibody-Dependent Cellular Cytotoxicity” or ‘ADCC”, refers to an immune mechanism through which Fc receptor-bearing effector cells can recognize and kill antibody-coated target cells expressing antigens recognized antibodies on their surface. More particularly, ADCC may refer the binding of an anti-CD127 agent (e.g. an antibody) to an epitope expressed on target cells and the subsequent Fc-dependent recruitment of effector immune cells expressing Fc receptors (essentially NK cells and activated lymphocytes), resulting in the killing of target cells mainly by granzyme/perforin-based mechanisms. An anti-CD127 agent that does not have an ADCC, in particular on immune cells, in particular on T cell, may be attributed to an agent (e.g. an antibody) which is able to induce a specific ADCC score strictly below 500 cpm using human NK cells as effector cells and radioactive chrome (Cr51) labeling to measure cell lysis. The ADCC assay may be carried out by co-culture of ⁵¹Cr labelled T cells (target cells) with human NK cells (effector cells) at a ratio of 10 NK for 1 T cell. Cytotoxicity (ADCC) may be evaluated by measurement of radioactive counts per minute (cpm) in the supernatant of this coculture after 4 hours of incubation at 37° C., 5% CO2.

As used herein, the term IL-7 signaling pathway is related to the intracellular molecular pathway induced when IL-7 binds to IL-7R and triggers the activation of several signaling pathways, including the Janus kinases (JAK)-1 and -3, signal transducer and activator of transcription 5 (STAT5) and phosphatidylinostol 3-kinase (PI3-k). The IL-7 signaling pathway may be considered to be activated when the phosphorylation of PI3-k and/or STAT5 and/or ERK is increased as compared to a negative control wherein the IL7-R is not stimulated (for example in absence of IL-7).

### Anti-CD127 Agents to be Used in the Method of the Invention or for Use According to the Invention

As used herein, an anti-CD127 agent refers to a compound selected from the list consisting of antibodies; antigen-binding fragments of an antibody; antigen-binding antibody mimetics; macromolecules comprising an antibody, an antigen-binding fragments of an antibody, or an antigen-binding antibody mimetics; and which binds, in particular specifically binds, to CD127, in particular human CD127, in particular human CD127 of SEQ ID No. 1, in particular to the extracellular domain of (human) CD127, most particularly to the extracellular domain of human CD127 of SEQ ID No. 2. The anti-CD127 agent to be used in the method of the invention, or for use according to the invention, has furthermore the capability to increase the Antibody Dependent Cellular Phagocytosis (ADCP) activity of CD127-positive tumor cells by macrophages, and does not have Antibody Dependent Cellular Cytotoxic (ADCC) activity, in particular on immune cells. In a particular embodiment of the invention, the anti-CD127 agent to be used in the method of the invention, or for use according to the invention, has the capability to increase the Antibody Dependent Cellular Phagocytosis (ADCP) activity of CD127-positive tumor cells by macrophages, and preferably does not have Antibody Dependent Cellular Cytotoxic (ADCC) activity, in particular on immune cells

An increase in the phagocytosis of CD127-positive tumor cells, in particular by macrophages, may be assessed by a comparison of the phagocytosis of CD127-positive tumor cells in two experiments, one experiment in presence of an anti-CD127 agent and one experiment in absence of the anti-CD127 agent. An increase in the phagocytosis of CD127-positive tumor cells, in particular by macrophages as compared to the same experiment in absence of the anti-CD127 agent may be considered when the phagocytosis is raised by at least 20%, preferably at least 30%; and most preferably at least 40%, as compared to the control.

The anti-CD127 agent used in the method of the invention of for use according to the invention shows an increased Antibody-Dependant Cellular Phagocytosis (ADCP) on CD127-positive cells, in particular CD127-positive tumor cells, in particular by macrophages. Antibody ADCP increase may be considered positive when specific phagocytosis against CD127-positive cells is superior by 20% in presence of the anti-CD127 agent as compared to the phagocytosis of the same CD127-positive cells in absence of the anti-CD127 agent. ADCP properties can be evaluated in an ADCP assay such as the test disclosed in the examples of the invention. More particularly, an ADCP assay may comprise the following steps: co-culturing for one hour leukemic cells labeled with a fluorescent dye and in presence of the anti-CD127 agent or in absence of the anti-CD127 agent with phagocytic cells, in particular macrophages, labeled with another fluorescent dye, and measuring the fluorescence of leukemic cells within phagocytic cells. The ADCP assay is preferably carried out in presence of human macrophages.

The anti-CD127 agent of the invention does not induce ADCC, in particular of immune cells, more particularly of T cells and/or of tumor cells. In particular, the ADCC potential of an anti-CD127 agent to be used according to the invention may be assessed according to the method disclosed here above or according to the examples of the invention, more particularly according to the method used in the example illustrated in FIG. 10B of the invention.

In a particular embodiment of the invention, the anti-CD127 agent is an antibody or a related compound, like but not limited to antigen-binding antibody fragment, and antigen-binding antibody mimetic, and has an enhanced antibody-dependent cell-mediated phagocytosis (ADCP) on CD127-positive cells and no Antibody-Dependent Cellular Cytotoxicity (ADCC), as compared to another antibody or related compound which does not increase ADCP on CD127-positive cells and/or increase ADCC activities, in particular on immune cells, more particularly on T cells, including some anti-CD127 antibodies that are able to recognize and bind to CD127, but do not have any enhancement capability on the antibody-dependent cell-mediated phagocytosis of CD127-positive tumor cells.

In a particular embodiment of the invention, the anti-CD127 agent is an anti-CD127 antibody, or an antigen-binding fragment thereof, that is a humanized antibody, and which comprises constant domains derived from human constant domains of antibodies.

In a particular embodiment of the invention, the anti-CD127 agent is an anti-CD127 antigen-binding fragment of an antibody that shares the same functions as a full anti-CD127 agent according to the invention that is an antibody, and has inferior or equal to 500, 400, 300, 200, 100 or 50 amino acids and have at least the capacity to bind CD127, the capacity to induce ADCP, and has not ADCC activity. In particular, an anti-CD127 antigen-binding fragment of an antibody according to the invention have a size from 80 to 200, in particular from 100 to 200, in particular from 80 to 160, in particular from 100 to 160 amino acids, and have at least the capacity to bind CD127.

In a particular embodiment of the invention, the anti-CD127 agent of the invention is a functional fragment of an anti-CD127 antibody. Functional equivalents of such an antibody include but are not limited to molecules that bind to CD127, with the proviso that these functional fragments have an ADCP capability and do not have ADCC capability. A suitable functional fragment might comprise, for example, a truncated form of a full antibody. Particularly, the functional equivalent consists of an amino acid sequence having at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% of identity with any anti-CD127 antibody or antigen-binding fragment thereof disclosed in the present description over their entire length. As used herein, the term “full antibody” refers to an antibody that is an anti-CD127 agent according to the invention for which the functional equivalent of the invention has similar function. The percentages of identity to which reference is made in the presentation of the present invention are determined on the basis of a global alignment of sequences to be compared, that is to say, on an alignment of sequences over their entire length, using for example the algorithm of Needleman and Wunsch 1970. This sequence comparison can be done for example using the needle software by using the parameter “Gap open” equal to 10.0, the parameter “Gap Extend” equal to 0.5, and a matrix “BLOSUM 62”. Software such as needle is available on the website ebi.ac.uk worldwide, under the name “needle”. Accordingly, the present invention provides a polypeptide, in particular a functional fragment of an antibody, which is an antagonist of CD127, which has antibody-dependent cell-mediated phagocytosis (ADCP) on CD127-positive cells, in particular by macrophages, and no Antibody-Dependent Cellular Cytotoxicity (ADCC), in particular on immune cells, more particularly on T cells, said polypeptide comprises consecutive amino acids having a sequence which has at least at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% of identity with antibody N13B2hVL6 constituted of the heavy chain of SEQ ID No. 27 and the light chain of SEQ ID No. 28.

In a particular embodiment, the antibody light chain constant domain is derived from a human kappa light chain constant domain.

More particularly, the antibody heavy chain constant domain is derived from a human IgG1, IgG2, IgG3, or IgG4 heavy chain constant region, particularly from IgG4 heavy chain constant region. “Derived from” means encompassing some punctual mutations by amino acid substitutions such as IgG4 (S228P) or IgG1 (E333A) (see Yang and Ambrogelly, Current Opinion in Biotechnology 2014 and Okasaki et al., J Mol Biol 2004). These mutations well known from the skilled person in the art, generally modify some parent chain properties. For example, they lead to less immunogenicity compared to the parental antibody or abrogate FcγReceptor binding or avoid dimerization of the monomer antibody or stabilize the dimerization rendering antibodies better for human therapeutic uses.

In a particular embodiment of the invention, the anti-CD127 agent to be used according to the method of the invention or for use according to the invention increases Antibody-Dependent Cellular Phagocytosis (ADCP) of CD127-positive cells, in particular CD127-positive tumor cells, in particular by macrophages, and thereof increases the phagocytosis of CD127-cancer cells in the host, the anti CD127 agent being an antibody or an antigen-binding fragment thereof which belongs to the class of IgG4 mammalian immunoglobulins, and has no ADCC activity. In a more particular embodiment of the invention, the anti-CD127 agent is an antibody or an antigen-binding fragment thereof, belongs to the class of IgG4 mammalian immunoglobulins.

In a particular embodiment of the invention, the anti-CD127 agent is an antagonist of IL7-R signaling pathway induced by the binding of IL7 to CD127. In other words, the anti-CD127 agent used in the method of the invention or for use according to the invention has the capability to disrupt or block the binding between IL-7 and CD127, as compared to the binding between IL-7 and CD127 in absence of the anti-CD127 agent. As used herein, a CD127 antagonist agent, in particular an anti-CD127 antagonist antibody or related compound, has its general meaning in the art and refers to any compound, natural or synthetic, that blocks, suppresses, or reduces the biological activity of IL-7. In particular, the CD127 antagonist inhibits the interactions between IL-7 and CD127. In particular, the CD127 antagonist inhibits or reduces the activation of the phosphatidylinositol 3-kinase and/or the ERK signaling pathway induced by IL-7. In the invention, it can be considered that an antibody (or antigen-binding fragment thereof) reduces, inhibits or blocks the binding of IL-7 to CD127 if said antibody (or antigen-binding fragment thereof) induces an increase superior to 1 log, preferably superior to 2 log, more preferably superior to 3 log, most preferably superior to 4 log, of the KD value of IL-7 to CD127 in a binding competitive assay by Blitz, as compared to the KD value of IL-7 to CD127 in presence of a control antibody (i.e. an antibody which does not specifically bind to IL-7 nor CD127).

In a particular embodiment, the anti-CD127 agent does not induce the activation of the phosphatidylinositol 3-kinase and/or the ERK signaling pathway and/or does not induce the phosphorylation of STAT5.

In a particular embodiment of the invention, the anti-CD127 agent is an anti-human CD127 antagonist antibody or an antigen-binding fragment thereof which comprises:

a VH chain comprising at least the following amino acid sequences:
VHCDR1 SEQ ID No. 3;
VHCDR2 SEQ ID No. 4;
VHCDR3 SEQ ID No. 5 or SEQ ID No. 6;
   and a VL chain comprising at least the following amino acid sequences:
VLCDR1 SEQ ID No. 7 or SEQ ID No. 8;
VLCDR2 SEQ ID No. 9 or SEQ ID No. 10;
VLCDR3 SEQ ID No. 11,
   said anti-CD127 antibody or an antigen-binding fragment thereof exhibiting ADCP activity against CD127-positive cells, in particular CD127-positive tumor cells. In an embodiment, the anti-human CD127 antibody or an antigen-binding fragment enhances the phagocytosis on CD127-positive cells, in particular CD127-positive tumor cells, by macrophages when administered to a patient, and has no ADCC activity, and is used for treating a patient having a cancer. Said anti-human CD127 antibody or an antigen-binding fragment is preferably an antagonist of the binding between IL-7 and CD127 as defined here above. In particular, said antibody or antigen-binding fragment thereof comprises a constant chain belonging to the subclass of IgG1, IgG2, IgG3 or IgG4, in particular the subclass of IgG4.

CDR domains have been identified according to the KABAT numbering.

In a particular embodiment, the anti-CD127 agent is an anti-human CD127 antagonist antibody or an antigen-binding fragment thereof which comprises:

a VH chain comprising at least the following amino acid sequences:
VHCDR1 SEQ ID No. 3;
VHCDR2 SEQ ID No. 4;
VHCDR3 SEQ ID No. 5;
   and a VL chain comprising at least the following amino acid sequences:
VLCDR1 SEQ ID No. 7;
VLCDR2 SEQ ID No. 9;
VLCDR3 SEQ ID No. 11.

In a particular embodiment of the invention, the anti-CD127 agent is an anti-human CD127 antagonist antibody or an antigen-binding fragment thereof which comprises:

a VH chain comprising at least the following amino acid sequences:
VHCDR1 SEQ ID No. 3;
VHCDR2 SEQ ID No. 4;
VHCDR3 SEQ ID No. 6;
   and a VL chain comprising at least the following amino acid sequences:
VLCDR1 SEQ ID No. 8;
VLCDR2 SEQ ID No. 10;
VLCDR3 SEQ ID No. 11.

In a particular aspect of the invention, the anti-CD127 agent is an anti-human CD127 antagonist antibody or an antigen-binding fragment thereof which comprises:

a heavy chain variable domain comprising or consisting of the amino acid sequence set forth in SEQ ID No. 12, SEQ ID No. 13, SEQ ID No. 14, SEQ ID No. 15 or SEQ ID No. 22, in particular SEQ ID No. 15 or SEQ ID No. 22; and
a light chain variable domain comprising or consisting of the amino acid sequence set forth in SEQ ID No. 16, SEQ ID No. 17, SEQ ID No. 18, SEQ ID No. 19, SEQ ID No. 23, SEQ ID No. 24, SEQ ID No. 25 or SEQ ID No. 26, in particular SEQ ID No. 19 or SEQ ID No. 26,
said anti-CD127 antibody or an antigen-binding fragment thereof exhibiting ADCP activity against CD127-positive cells, in particular CD127-positive tumor cells, and has no ADCC activity. In an embodiment, the anti-human CD127 antibody or an antigen-binding fragment enhances the phagocytosis of CD127-positive cells, in particular CD127-positive tumor cells, by macrophages when administered to a patient, and is used for treating a patient having a cancer. Said anti-human CD127 antibody or an antigen-binding fragment is preferably an antagonist of the binding between IL-7 and CD127 as defined here above. In particular, said antibody or antigen-binding fragment thereof comprises a constant chain belonging to the subclass of IgG1, IgG2, IgG3 or IgG4, in particular the subclass of IgG4.

In a particular embodiment, the heavy chain variable domain is linked to the constant heavy chain consisting of the sequence of SEQ ID No: 30, to constitute a complete antibody heavy chain.

In a particular embodiment, the light chain variable domain is linked to the constant light chain consisting of a sequence selected from SEQ ID No: 31 and SEQ ID No: 32, in particular SEQ ID No: 31, to constitute a complete antibody light chain.

In a particular embodiment, the anti-CD127 agent is an anti-human CD127 antagonist antibody or an antigen-binding fragment thereof which comprises the heavy chain variable domain comprising or consisting of the amino acid sequence set forth in SEQ ID No. 12, and the light chain variable domain comprising or consisting of the amino acid sequence set forth in SEQ ID No. 16.

In a particular embodiment, the anti-CD127 agent is an anti-human CD127 antagonist antibody or an antigen-binding fragment thereof which comprises the heavy chain variable domain comprising or consisting of the amino acid sequence set forth in SEQ ID No. 15, and the light chain variable domain comprising or consisting of the amino acid sequence set forth in SEQ ID No. 19.

In a particular embodiment, the anti-CD127 agent is an anti-human CD127 antagonist antibody or an antigen-binding fragment thereof which comprises the heavy chain variable domain comprising or consisting of the amino acid sequence set forth in SEQ ID No. 22, and the light chain variable domain comprising or consisting of the amino acid sequence set forth in SEQ ID No. 26.

In a particular aspect of the invention, the anti-CD127 agent is an anti-human CD127 antagonist antibody or an antigen-binding fragment thereof which comprises:

a heavy chain comprising or consisting of the amino acid sequence set forth in SEQ ID No. 20 or SEQ ID No. 27, and a light chain comprising or consisting of the amino acid sequence set forth in SEQ ID No. 21, SEQ ID No. 28 or SEQ ID No. 29,
said anti-CD127 antibody or an antigen-binding fragment thereof exhibiting ADCP activity against CD127-positive cells, in particular CD127-positive tumor cells, and has no ADCC activity. In an embodiment, the anti-human CD127 antibody or an antigen-binding fragment enhances the phagocytosis of CD127-positive cells, in particular CD127-positive tumor cells, by macrophages when administered to a patient, and is used for treating a patient having a cancer. Said anti-human CD127 antibody or an antigen-binding fragment is preferably an antagonist of the binding between IL-7 and CD127 as defined here above. In particular, said antibody or antigen-binding fragment thereof comprises a constant chain belonging to the subclass of IgG1, IgG2, IgG3 or IgG4, in particular the subclass of IgG4.

In a particular aspect of the invention, the anti-CD127 agent is an anti-human CD127 antagonist antibody or an antigen-binding fragment thereof which comprises:

a heavy chain comprising or consisting in the amino acid sequence set forth in SEQ ID No. 27 and a light chain comprising or consisting of the amino acid sequence set forth in SEQ ID No. 28 or SEQ ID No. 29, in particular a heavy chain comprising or consisting in the amino acid sequence set forth in SEQ ID No. 27 and a light chain comprising or consisting of the amino acid sequence set forth in SEQ ID No. 28,
said anti-CD127 antibody or an antigen-binding fragment thereof exhibiting ADCP activity against CD127-positive cells, in particular CD127-positive tumor cells, and has no ADCC activity. In an embodiment, the anti-human CD127 antibody or an antigen-binding fragment enhances the phagocytosis of CD127-positive cells, in particular CD127-positive tumor cells, by macrophages when administered to a patient, and is used for treating a patient having a cancer. Said anti-human CD127 antibody or an antigen-binding fragment is preferably an antagonist of the binding between IL-7 and CD127 as defined here above. In particular, said antibody or antigen-binding fragment thereof comprises a constant chain belonging to the subclass of IgG1, IgG2, IgG3 or IgG4, in particular the subclass of IgG4.

Table 1 details different combinations of amino acid sequences corresponding to several anti-CD127 agent used in the examples of the invention. It should be noted that this table is merely for illustrative purpose, and anti-CD127 agents disclosed herein should not be considered as the sole anti-CD127 agent that can be used according to the invention, or in a method according to the invention.

TABLE 1
Sequences SEQ ID No. of the CDRs, heavy and light chain
variable domains, and full heavy and light chains of several
antibodies used in the examples of the invention.

| Antibody | N13B2 | N13B2hVL6 | N12B3h3 |
|---|---|---|---|
| CDRs SEQ ID No. | SEQ ID No. | SEQ ID No. | SEQ ID No. |
| (HCDR1/HCDR2/ | 3/4/5/7/9/11 | 3/4/6/8/10/11 | 3/4/6/8/10/11 |
| HCDR3/LCDR1/ | | | |
| LCDR2/LCDR3) | | | |
| Heavy chain variable | SEQ ID No. 12 | SEQ ID No. 22 | SEQ ID No. 20 |
| domain SEQ ID No. | | | |
| Light chain variable | SEQ ID No. 16 | SEQ ID No. 26 | SEQ ID No. 21 |
| domain SEQ ID No. | | | |
| Full heavy chain | SEQ ID No. 12 + | SEQ ID No. 27 | SEQ ID No. 20 + |
| SEQ ID No. | SEQ ID No. 30 | | SEQ ID No. 30 |
| Full light chain | SEQ ID No. 16 + | SEQ ID No. 28 | SEQ ID No. 21 + |
| SEQ ID No. | SEQ ID No. 31 | | SEQ ID No. 31 |

| Antibody | N13B2hVL3 | N13B2hVL4 | N13B2hVL5 |
|---|---|---|---|
| CDRs SEQ ID No. | SEQ ID No. | SEQ ID No. | SEQ ID No. |
| (HCDR1/HCDR2/ | 3/4/6/8/10/11 | 3/4/6/8/10/11 | 3/4/6/8/10/11 |
| HCDR3/LCDR1/ | | | |
| LCDR2/LCDR3) | | | |
| Heavy chain variable | SEQ ID No. 22 | SEQ ID No. 22 | SEQ ID No. 22 |
| domain SEQ ID No. | | | |
| Light chain variable | SEQ ID No. 23 | SEQ ID No. 24 | SEQ ID No. 25 |
| domain SEQ ID No. | | | |
| Full heavy chain | SEQ ID No. 22 + | SEQ ID No. 22 + | SEQ ID No. 22 + |
| SEQ ID No. | SEQ ID No. 30 | SEQ ID No. 30 | SEQ ID No. 30 |
| Full light chain | SEQ ID No. 23 + | SEQ ID No. 24 + | SEQ ID No. 25 + |
| SEQ ID No. | SEQ ID No. 31 | SEQ ID No. 31 | SEQ ID No. 31 |

In a particular embodiment of the invention, the anti-CD127 agent to be used according to the invention or for use in a method according to the invention is provided as an isolated nucleic acid molecule or group of isolated nucleic acid molecules encoding an anti-CD127 agent as defined herein according to the invention, in particular an antibody or an antigen-binding fragment thereof. Particularly, said nucleic acid molecule or group of nucleic acid molecule encodes the light chain variable domain or the light chain of an antibody provided herein, and the heavy chain variable domain or heavy chain of an antibody provided herein, according to any of the definitions provided herein. In particular, the isolated nucleic acid molecules or the group of isolated nucleic acid molecules encodes:
a heavy chain variable domain comprising or consisting of the amino acid sequence set forth in SEQ ID No. 12, SEQ ID No. 13, SEQ ID No. 14, SEQ ID No. 15 or SEQ ID No. 22, in particular SEQ ID No. 15 or SEQ ID No. 22; and
a light chain variable domain comprising or consisting of the amino acid sequence set forth in SEQ ID No. 16, SEQ ID No. 17, SEQ ID No. 18, SEQ ID No. 19, SEQ ID No. 23, SEQ ID No. 24, SEQ ID No. 25 or SEQ ID No. 26, in particular SEQ ID No. 19 or SEQ ID No. 26.

More particularly, the isolated nucleic acid molecules or the group of isolated nucleic acid molecules encodes a heavy chain comprising or consisting of the amino acid sequence set forth in SEQ ID No. 20 or SEQ ID No. 27, and a light chain comprising or consisting of the amino acid sequence set forth in SEQ ID No. 21, SEQ ID No. 28 or SEQ ID No. 29.

### Diseases to be Treated

The patients to be treated according to a method of the invention have, develop or are likely to develop a CD127-positive cancer. A CD127-positive cancer is a cancer wherein tumor cells express the Cluster of Differentiation 127 (CD127). “CD127-positive tumor cell” designates a tumor cell expressing CD127 at their cell surface. In most cases, CD127-positive cell expresses CD127 in a complex forming the IL-7R (IL-7R-positive cells) and/or in a complex forming the TSLPR (TSLPR-positive cells). A cancer may be classified in the subset of CD127-positive cancer by flow cytometry evaluation using a primary mouse monoclonal antibody directed against CD127 (mouse monoclonal anti-human CD127 clone [A019D5], cat. 351304, Biolegend) and an irrelevant monoclonal mouse antibody (for example mouse monoclonal immunoglobulin IgG1, k, isotype control, cat.555746, BD Pharmingen) and a kit for quantitative determination of cell surface antigens, such as BD Quantibrite™ Beads (cat. 340495 BD Pharmingen): a CD127-positive cancer is defined by a Specific Antibody-Binding Capacity (SABC) strictly superior to zero. A CD127-positive cancer overexpressing CD127 is considered when CD127 expression level as measured by flow cytometry or RNA sequencing is higher than on healthy T cells or normal bone marrow control cells. In a particular embodiment of the invention, the patient to be treated has Leukemia. Leukemia (also written “leukaemia”) is a malignant progressive disease in which the bone marrow and other blood-forming organs produce increased numbers of immature or abnormal leucocytes. These suppress the production of normal blood cells, leading to anemia and other symptoms. Leukemia are also referenced as “liquid cancers” or “blood cancers”. In the present description, the three terms “leukemia”, “liquid cancer” and “blood cancer” share the same meaning, except when expressly stated otherwise.

In a particular embodiment of the invention, the patient to be treated has an acute lymphoblastic leukemia. Acute lymphoblastic leukemia (ALL) is a cancer of the lymphoid line of blood cells. The lymphoid cell line is a type of white blood cell also referenced as Lymphocytes. Lymphocytes include natural killer cells (which function in cell-mediated, cytotoxic innate immunity), T cells (for cell-mediated, cytotoxic adaptive immunity), and B cells (for humoral, antibody-driven adaptive immunity).

In a particular embodiment of the invention, the patient to be treated has T-cell leukemia or T-cell ALL, in particular T-cell ALL.

In a particular embodiment of the invention, the patient to be treated has B-cell leukemia or B-cell ALL, in particular B-cell ALL.

T-cell ALL is a cancer more particularly related to the provision of immature or abnormal T cells. B-cell ALL is a cancer more particularly related to the provision of immature or abnormal B cells.

In a particular embodiment of the invention, the patient to be treated has a ALL selected from the following group: CD127 overexpressing ALL (which can be determined by comparing the CD127 expression in ALL cells and in healthy bone marrow cells), CD127 and/or JAK-STAT pathway mutated ALL (as compared to healthy cells), including BCR-ABL1-like ALL, as well as B cell precursor ALL bearing the following cytogenetics: t(1;19), t(12,21), MLL-rearrangements, Hyperdiploid karyotypes, trisomy 4 and trisomy 10. In a particular embodiment, the invention relates to anti-CD127 antibodies or antigen-binding fragments thereof, having (i.e. increasing as compared to a negative control) Antibody Dependent Cellular Phagocytosis (ADCP) activity macrophages targeting CD127-positive cancer cells, in particular wherein said activity is achieved by or involves macrophages, for the treatment of ALL selected from the group consisting of CD127 wild type T-ALL (HPB-ALL cell line), CD127-mutated T-ALL (DND41 cell line), t(1;19) B-ALL (697 cell line), t(12;21) B-ALL (REH cell line) and t(5;12) B-ALL (NALM6 cell line).

### Combinations of Agents

In an embodiment of the invention, the anti-CD127 agent for use in the method of the invention or for use according to the invention is administered to the patient in combination with a second active ingredient, like another therapeutic agent. Said second active ingredient includes but is not limited to probiotics and therapeutic agents as described below.

The present invention thus also relates to the combination of an anti-CD127 agent for use in the method of the invention or for use according to the invention, in combination with a second active ingredient. The combination is for use in the treatment of CD127-positive cancer by enhancing the phagocytosis of CD127-positive tumor cells. The second active ingredient does not necessarily have any effect on the phagocytosis of CD127-positive tumor cells, but may have other properties useful in the treatment of cancer.

In some embodiments, the anti-CD127 agent for use in the method of the invention or for use according to the invention is administered to the patient in combination with a standard (conventional) treatment. The present invention relates thus to the combination of an anti-CD127 agent for use in the method of the invention or for use according to the invention with a conventional treatment for use in the treatment of cancer. As used herein, the term “standard or conventional treatment” refers to any treatment of cancer (drug, radiotherapy, etc) usually administrated to a patient who suffers from cancer.

In some embodiments, the anti-CD127 agent for use in the method of the invention or for use according to the invention is administered to the subject in combination with at least one further therapeutic agent, e.g. for treating cancers. Such administration may be simultaneous, separate or sequential. For simultaneous administration, the agents may be administered as one composition or as separate compositions, as appropriate. The further therapeutic agent is typically relevant for the disorder to be treated. Exemplary therapeutic agents include other anti-cancer antibodies, cytotoxic agents, chemotherapeutic agents, anti-angiogenic agents, anti-cancer immunogens, cell cycle control/apoptosis regulating agents, hormonal regulating agents, and other agents described below.

In some embodiments, the anti-CD127 agent for use in the method of the invention or for use according to the invention is used in combination with a chemotherapeutic agent, a targeted cancer therapy, an immunotherapeutic agent or radiotherapy.

In some embodiments, the anti-CD127 agent for use in the method of the invention or for use according to the invention is used in combination with a chemotherapeutic agent or targeted therapy agent. The present invention relates thus to the combination of an antagonist of CD127 with a chemotherapeutic agent or targeted therapy agent for use in the treatment of CD127-positive cancer.

The second therapeutic agent may be selected from the list consisting of anti-CD3 agent, in particular anti-CD3 antibody, anti-CD19 agent, in particular an anti-CD19 antibody, and anti-CD47 agent, in particular an anti-CD47 antibody, more particularly an anti-CD47 antagonist agent, even more particularly an anti-CD47 antagonist antibody, an inhibitor of the tyrosine/kinase pathway, Dexamethasone, rituximab, trastuzumab, cetuximab. Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Doxorubicin Hydrochloride; Gleevec (Imatinib Mesylate); Iclusig (Ponatinib Hydrochloride); Inotuzumab Ozogamicin; Imatinib Mesylate; Kymriah (or Tisagenlecleucel); Marqibo (Vincristine Sulfate Liposome); Mercaptopurine (or Purinethol or Purixan); Methotrexate Sodium (or Trexall); Nelarabine; Oncaspar (or Pegaspargase); Ponatinib Hydrochloride; Prednisone; Purinethol (Mercaptopurine); Vincristine Sulfate or Vincristine Sulfate Liposome. In a particular embodiment of the invention, the second therapeutic agent is selected from the list consisting of Dexamethasone, an anti-CD47 antagonist antibody, an inhibitor of the tyrosine kinase pathway.

As used herein, an anti-CD47 antagonist agent, in particular an anti-CD47 antagonist antibody, has its general meaning in the art and refers to any compound, natural or synthetic, that blocks, suppresses, or reduces the biological activity of CD47. In particular, the CD47 antagonist inhibits the interactions between CD47 and one of its ligand, in particular SIRPa.

An inhibitor of the tyrosine/kinase pathway is a pharmaceutical drug that inhibits tyrosine kinases. These inhibitors are usually also called tyrphostins. These inhibitors usually either compete with adenosine triphosphate (ATP), the phosphorylating entity of the tyrosine kinase, the substrate of the tyrosine kinase, or modify the confirmation of the tyrosine kinase, thereby modifying its phosphorylating activity.

In a particular embodiment if the invention, it is provided a combination of compounds comprising:
a) an anti-CD127 agent, in particular an anti-CD127 antibody or antigen-binding fragment thereof or antigen-binding antibody mimetic, that has Antibody Dependent Cellular Phagocytosis (ADCP) activity on CD127-positive tumor cells, in particular by macrophages, and has no ADCC activity; and
b) dexamethasone and/or an anti-CD47 antagonist antibody, and/or an inhibitor of the tyrosine/kinase pathway, in particular dexamethasone.

In a particular embodiment if the invention, it is provided a combination of compounds comprising:
a) an anti-human CD127 antagonist antibody or an antigen-binding fragment thereof which comprises:
   a VH chain comprising at least the following amino acid sequences:
VHCDR1 SEQ ID No. 3;
VHCDR2 SEQ ID No. 4;
VHCDR3 SEQ ID No. 5 or SEQ ID No. 6;
   and a VL chain comprising at least the following amino acid sequences:
VLCDR1 SEQ ID No. 7 or SEQ ID No. 8;
VLCDR2 SEQ ID No. 9 or SEQ ID No. 10;
VLCDR3 SEQ ID No. 11,
   said anti-CD127 antibody or an antigen-binding fragment thereof exhibiting ADCP activity against CD127-positive cells, in particular CD127-positive tumor cells, in particular by macrophages, and has no ADCC activity; and
b) dexamethasone and/or an anti-CD47 antagonist antibody, and/or an inhibitor of the tyrosine/kinase pathway, in particular dexamethasone.

In a particular embodiment, the anti-human CD127 antagonist antibody present in the combination of compounds comprises:

a VH chain comprising at least the following amino acid sequences:
VHCDR1 SEQ ID No. 3;
VHCDR2 SEQ ID No. 4;
VHCDR3 SEQ ID No. 5;
   and a VL chain comprising at least the following amino acid sequences:
VLCDR1 SEQ ID No. 7;
VLCDR2 SEQ ID No. 9;
VLCDR3 SEQ ID No. 11.

In a particular embodiment, the anti-human CD127 antagonist antibody present in the combination of compounds comprises:

a VH chain comprising at least the following amino acid sequences:
VHCDR1 SEQ ID No. 3;
VHCDR2 SEQ ID No. 4;
VHCDR3 SEQ ID No. 6;
   and a VL chain comprising at least the following amino acid sequences:
VLCDR1 SEQ ID No. 8;
VLCDR2 SEQ ID No. 10;
VLCDR3 SEQ ID No. 11.

In a particular aspect of the invention, the combination of compounds comprises:

i) an anti-CD127 agent which is an anti-human CD127 antagonist antibody or an antigen-binding fragment thereof which comprises:
a heavy chain variable domain comprising or consisting of the amino acid sequence set forth in SEQ ID No. 12, SEQ ID No. 13, SEQ ID No. 14, SEQ ID No. 15 or SEQ ID No. 22, in particular SEQ ID No. 15 or SEQ ID No. 22; and
a light chain variable domain comprising or consisting of the amino acid sequence set forth in SEQ ID No. 16, SEQ ID No. 17, SEQ ID No. 18, SEQ ID No. 19, SEQ ID No. 23, SEQ ID No. 24, SEQ ID No. 25 or SEQ ID No. 26, in particular SEQ ID No. 19 or SEQ ID No. 26;
and
ii) dexamethasone and/or an anti-CD47 antagonist antibody, and/or an inhibitor of the tyrosine/kinase pathway, in particular dexamethasone.

In a particular embodiment, the anti-human CD127 antagonist antibody present in the combination of compounds comprises:

the heavy chain variable domain comprising or consisting of the amino acid sequence set forth in SEQ ID No. 12, and the light chain variable domain comprising or consisting of the amino acid sequence set forth in SEQ ID No. 16; or
the heavy chain variable domain comprising or consisting of the amino acid sequence set forth in SEQ ID No. 15, and the light chain variable domain comprising or consisting of the amino acid sequence set forth in SEQ ID No. 19; or
the heavy chain variable domain comprising or consisting of the amino acid sequence set forth in SEQ ID No. 22, and the light chain variable domain comprising or consisting of the amino acid sequence set forth in SEQ ID No. 26.

In a particular aspect, the invention, the combination of compounds comprises:
an anti-CD127 agent which is an anti-human CD127 antagonist antibody or an antigen-binding fragment thereof which comprises:
   a heavy chain comprising or consisting of the amino acid sequence set forth in SEQ ID No. 20 or SEQ ID No. 27 and a light chain comprising or consisting of the amino acid sequence set forth in SEQ ID No. 21, SEQ ID No. 28 or SEQ ID No. 29;
   and
dexamethasone and/or an anti-CD47 antagonist antibody, and/or an inhibitor of the tyrosine/kinase pathway, in particular dexamethasone.

In a particular embodiment, the anti-human CD127 antagonist antibody present in the combination of compounds comprises or consists a heavy chain comprising or consisting in the amino acid sequence set forth in SEQ ID No. 27 and a light chain comprising or consisting of the amino acid sequence set forth in SEQ ID No. 28 or SEQ ID No. 29, in particular a heavy chain comprising or consisting in the amino acid sequence set forth in SEQ ID No. 27 and a light chain comprising or consisting of the amino acid sequence set forth in SEQ ID No. 28.

### Particular Methods for Treating CD127-positive Cancers

In some embodiments, the method of the invention or the use of an anti-CD127 agent as described herein is for treating a patient having a CD127-positive cancer and who has deleterious side effects associated to the treatment of its cancer by a conventional treatment, such as chemotherapy and/or administration of an anti-CD3, anti-CD19 and/or andi-CD47 compound. Deleterious side effect may include cytokine release syndrome, severe neuroxicity, sinusoidal obstruction syndrome, hepatic toxicity, lymphodepletion.

In a particular embodiment, the method of the invention or the use of the anti-CD127 agent as described herein is for treating a patient having a cancer as a complementary treatment, in particular ALL, in particular a CD127-positive cancer, the patient being or having been treated with a first treatment, in particular chemotherapy, chemotherapy with stem cell transplant, radiation therapy, surgery and/or immunotherapy.

In a particular embodiment, the method of the invention or the use of the anti-CD127 agent as described herein is for treating a patient having a cancer as a complementary treatment, in particular ALL, in particular a CD127-positive cancer, the method of the use comprising a first step of determining if the patient has a CD127-positive cancer, in particular if the patient has a CD127-positive ALL.

In a particular embodiment, the method of the invention or the use of the anti-CD127 agent as described herein is for treating a patient having an ALL and having a lymphodepletion due to a treatment with a conventional treatment of ALL.

In a particular embodiment, the method of the invention or the use of the anti-CD127 agent as described herein is for treating a patient having an ALL, and before metastatic cells issued from the ALL pass within the central nervous system. The use of the anti-CD127 agent may indeed prevent metastatic cells to spread to the central nervous system, on the contrary to conventional treatment of ALL.

In a particular embodiment, the method of the invention or the use of the anti-CD127 agent as described herein is for treating a patient having an ALL with metastatic cells, said metastatic cells having not reached the central nervous system. The use of the anti-CD127 agent may indeed prevent metastatic cells to spread to the central nervous system, on the contrary to conventional treatment of ALL

In a particular embodiment, the method of the invention or the use of the anti-CD127 agent as described herein is for treating a patient having an ALL and who cannot be treated with a conventional treatment of ALL, in particular due to the toxicity of the conventional treatment or due to the non-responding status of the patient to such treatment or to acquired resistance to such treatment.

In a particular embodiment, the method of the invention or the use of the anti-CD127 agent as described herein is for treating a patient having an ALL, and who does not positively respond to a treatment with a conventional treatment of ALL, in particular patient who does not respond to at least one of the following drug: an anti-CD3 agent, in particular anti-CD3 antibody, an anti-CD19 agent, in particular an anti-CD19 antibody, and an anti-CD47 agent, in particular an anti-CD47 antibody, in particular an anti-CD47 antagonist agent, in particular an anti-CD47 antagonist antibody, an inhibitor of the tyrosine/kinase pathway, Dexamethasone, rituximab, trastuzumab, cetuximab Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Dexamethasone; Doxorubicin Hydrochloride; Gleevec (Imatinib Mesylate); Iclusig (Ponatinib Hydrochloride); Inotuzumab Ozogamicin; Imatinib Mesylate; Kymriah (or Tisagenlecleucel); Marqibo (Vincristine Sulfate Liposome); Mercaptopurine (or Purinethol or Purixan); Methotrexate Sodium (or Trexall); Nelarabine; Oncaspar (or Pegaspargase); Ponatinib Hydrochloride; Prednisone; Purinethol (Mercaptopurine); Vincristine Sulfate or Vincristine Sulfate Liposome. In a particular embodiment, the patient does not positively respond to a treatment with a compound selected from the list consisting of an anti-CD47 antagonist antibody, an inhibitor of the tyrosine/kinase pathway and Dexamethasone, in particular Dexamethasone.

In some embodiments, the method of the invention or the use of an anti-CD127 agent as described herein is for treating a patient having a CD127-positive cancer and who has not been treated yet with a conventional treatment known for having deleterious side effects, such as chemotherapy and/or administration of an anti-CD3 compound and/or an anti-CD19 compound and/or administration of an anti-CD47 compound, and/or administration of dexamethasone, and/or an inhibitor of the tyrosine/kinase pathway.

In some embodiments, the method of the invention or the use of an anti-CD127 agent as described herein is for treating a patient having a CD127-positive cancer and who is resistant to a conventional treatment, such as administration of dexamethasone.

In some embodiments, the method of the invention or the use of an anti-CD127 agent as described herein is for treating a patient having a CD127-positive cancer which has not metastased yet.

In some embodiments, the method of the invention or the use of an anti-CD127 agent as described herein is for treating a patient having a CD127-positive cancer, the patient being a child (i.e. being less than 15 year old).

### Method for Selecting a Patient to be Treated with an Anti-CD127 Agent in Replacement of a Treatment with an Anti-CD19 Agent, Like Blinatumomab, or Dexamethasone

In an embodiment of the invention, it is provided a method to select a patient having a CD127-positive cancer, in particular an ALL, more particularly T cell ALL or B cell ALL, and most particularly CD127 overexpressing Acute Lymphoblastic Leukemia (ALL), CD127 and/or JAK-STAT pathway mutated ALL, BCR-ABL1-like ALL, and B cell precursor ALL bearing one the following cytogenetics: t(1;19), t(12,21), MLL-rearrangements, hyperdiploid karyotypes, trisomy 4 and trisomy 10, and who can be treated by administration of an anti-CD127 agent as defined in the present description, the method comprising the measurement of the expression of CD127 by tumor cells, and the measurement of the expression of CD19 by tumor cells, a patient being able to be treated by the administration of an anti-CD127 agent when i) the tumor cells express CD127 and ii) the tumor cells do not express CD19, meaning that the ALL is resistant to a treatment by an anti-CD19 agent, like Blinatumomab.

In an embodiment of the invention, it is provided a method to select a patient having a CD127-positive cancer, in particular an ALL, more particularly T cell ALL or B cell ALL, and most particularly CD127 overexpressing Acute Lymphoblastic Leukemia (ALL), CD127 and/or JAK-STAT pathway mutated ALL, BCR-ABL1-like ALL, and B cell precursor ALL bearing one the following cytogenetics: t(1;19), t(12,21), MLL-rearrangements, hyperdiploid karyotypes, trisomy 4 and trisomy 10, and who can be treated by administration of an anti-CD127 agent as defined in the present description, the method comprising the measurement of resistance of the tumor cells to a treatment with dexamethasone, and when the tumor cells are resistant to dexamethasone, the method further comprise the measurement of the resistance of the tumor cells to a treatment with an anti-CD127 agent as described herein, alone or in combination with dexamethasone.

### Method for Determining the Likelihood of a Patient to Respond to a Treatment with an Anti-CD127 Agent

In an embodiment the invention relates to a method of determining the likelihood of a patient diagnosed with cancer to benefit from a treatment with an anti-CD127 agent, wherein a biological sample, in particular a blood sample, previously obtained from the patient is assayed for the presence of CD127 positive cells and wherein in case such cells are contained in the sample the patient's condition is considered likely to benefit from a treatment with an anti-CD127 agent as described herein.

### LEGENDS OF THE FIGURES

FIG. 1. Minimal residual disease (MRD) eradication in patients derived xenografts (PDX) experiments. (A) and (B) correspond to the probability of survival of mice over days post-transplant of two PDX issued from two different pediatric patients having t(1;19) ALL. Mice are treated with two different anti-CD127 agents with ADCP capabilities (an antagonist anti-CD127 agent in red (N13B2-hVL6) and a neutral (i.e. not antagonist nor agonist) anti-CD127 agent in green, Effi-3-VH3VL3), and a negative control (blue).

FIG. 2. Overt leukemia development in patients derived xenografts (PDX) experiments. (A) and (B) correspond to the probability of survival of mice over days post-transplant of two overt leukemia PDX issued from two different pediatric patients having t(1;19) ALL. Results illustrated in (A1) and (B1) correspond to mice treated with an antagonist anti-CD127 agent with ADCP capabilities (red, N13B2-hVL6) and a negative control (blue). Results illustrated in (A2) and (B2) correspond to mice treated with a neutral (i.e. not antagonist nor agonist) anti-CD127 agent with ADCP capabilities (green, Effi-3-VH3VL3) and a negative control (blue).

FIG. 3. Quantification of specific antibody binding of N13B2-hVL6 in a panel of tumor cell lines issued from different kinds of Acute Lymphoblastic Leukemias. Jurkat, HPB-ALL and DND41 correspond to three different T-cell ALL cell lines. 697, NALM6 and REH correspond to three different B-cell ALL cell lines. The specific binding N13B2-hVL6 was evaluated as the fold change of fluorescence intensity compared to that of an isotype control on each cell line.

FIG. 4. Normalized phagocytic index of tumor cells issued from different kinds of Acute Lymphoblastic Leukemia cell lines treated with an antagonist anti-CD127 agent (N13B2-hVL6).

FIG. 5. Phagocytosis of leukemia cells in a sample treated with N13B2-hVL6 with ADCP+ ADCC− capabilities as compared to a control. Leukemia cells (NALM6 cell line) are colored in red, while human M1 macrophages are colored in green. White arrows point to macrophages phagocytosing leukemia cells.

FIG. 6. Normalized phagocytosis index of leukemia cells in T-ALL models. Two T-ALL cell lineages (HPB-ALL on the left and DND41 IL7-R mutated on the right) have been treated with increasing doses of anti-CD127 antibodies (N13B2-hVL6, 1A11, and EFFI-3-VH3VL3).

FIG. 7. Normalized phagocytosis index of leukemia cells in B-ALL models. Three B-ALL cell lineages (697 t(1,19) BCP-ALL on the left, NALM6 DUX4 BCP-ALL in the middle and REH t(12;21) BCP-ALL on the right) have been treated with increasing doses of anti-CD127 antibodies (N13B2-hVL6, 1A11, HAL and EFFI-3-VH3VL3).

FIG. 8. Toxicity of anti-CD127 antibodies and anti-CD47 antibodies on macrophages. The viability of macrophages treated with an anti-CD127 antibody (N13B2-hVL6) or an anti-CD47 antibody (5F9) has been assessed with increasing doses of antibodies.

FIG. 9. Therapeutic windows for treating ALL with an anti-CD127 antibody or an anti-CD47 antibody. The phagocytic index of normal T cells versus diseased cells (REH model of B-ALL) has been compared in samples treated with increasing doses of an anti-CD127 antibody (N13B2-hVL6) or an anti-CD47 antibody (5F9).

FIG. 10. A. Phagocytosis of macrophages by macrophages (termed here “autophagocytosis” when treated with an anti-CD47 antibody (5F9), an anti-CD127 antibody (N13B2-hVL6) as compared to a negative control antibody (hlgG4). B. ADCC of human T cells by Natural Killer cells (NK) in presence of an anti-CD127 agent to be used according to the invention (N13B2-hVL6) and a positive control (an anti-CD127 antibody that is known to have an ADCC activity).

FIG. 11. Number of lymphocytes in the blood of healthy volunteers treated with N13B2-hVL6. (A) and (B) correspond to the lymphocyte counts measured in blood samples collected from healthy volunteers participating to the Single Ascending Dose cohort (SAD, 1 intra-venous injection) and to the Multiple Ascending Dose cohort (MAD, 2 intra-venous injections, 15 days apart), respectively.

FIG. 12. A. Example of resistance of T-ALL cells to Dexamathasone (HPB-ALL cell line, 48 h treatment). B. Induction of CD127 expression in a Dexamethasone dose-dependent manner in HPB-ALL cells (48 h treatment).

FIG. 13. Phagocytosis index of HPB-ALL T-ALL cells in response to N13B2-hVL6 with (red triangles) or without (black dots) Dexamethasone treatment (10 μM for 48 h).

FIG. 14. Phagocytosis index of leukemia cells treated with several anti-CD127 antibodies as defined in the present description. (A) on BCP-ALL cell line and (B) on REH t(12;21) BCP-ALL cell line. NB13B2-hVL6, VL3, VL4, VL5 and N13B2-h3 share the same subset of CDR domains (corresponding to HCDR1 of SEQ ID No.: 3; HCDR2 of SEQ ID No.: 4; HCDR3 of SEQ ID No.: 6; LCDR1 of SEQ ID No.: 8; LCDR2 of SEQ ID No.: 10 and LCDR3 of SEQ ID No.: 11) but have different framework sequences. N13B2-hVL6 has the heavy chain variable domain of SEQ ID No. 22, and the light chain variable domain of SEQ ID No. 26; VL2 has the heavy chain variable domain of SEQ ID No. 22, and the light chain variable domain of SEQ ID No. 21; VL3 has the heavy chain variable domain of SEQ ID No. 22, and the light chain variable domain of SEQ ID No. 23; VL4 has the heavy chain variable domain of SEQ ID No. 22, and the light chain variable domain of SEQ ID No. 24; VL5 has the heavy chain variable domain of SEQ ID No. 22, and the light chain variable domain of SEQ ID No. 25. N13B2alpha and beta are chimeric anti-CD127 antibodies which share closely related CDRs domains (with only one or two mutations within the 6 CDRs domains) with N13B2-hVL6 (CDRs of sequences SEQ ID No. 3, SEQ ID No. 4, SEQ ID No. 5, SEQ ID No. 7, SEQ ID No. 9 and SEQ ID No. 11).

FIG. 15. Binding of N13B2-hVL6 and control ADCP+/ADCC+ anti-CD127 antibody to main FCγRs, namely A. CD16a, B. CD32a and C. CD64 assessed by ELISA.

### EXAMPLES

### Material and Methods

ALL patient samples, human leukemic cell lines. Leukemia patients were treated according to ALL-Berlin-Frankfurt-Münster (BFM) 2000 or 2009 protocols after informed consent in accordance with the Declaration of Helsinki. The study was approved by the ethical committee of the Christian-Albrechts-University Kiel (D437/17). Jurkat, HPB-ALL and DND41 T-ALL cell lines were purchased from ATCC. 697, NALM6 and REH B-ALL cell lines were purchased from DSMZ (Leibniz Institute, Germany). All cells were tested and found free from mycoplasma.

Mice. NOD.Cg-Prkdcscid Il2rgtm1Wjl/SzJ mice expressing Hc (NSG-Hc) mice were generated by backcrossing the intact Hc gene from the NOD-CBALs-Hc1/Lt congenic strain into the NSG strain, in collaboration with Lenny Shultz (Jackson Laboratories, Bar Harbor, USA). NSG-Hc mice were bred under pathogen-free conditions at Schleswig-Holstein Kiel University and xenografts were generated in accordance with governmental regulations (Schleswig-Holstein Ministerium für Energiewende, Landwirtschaft, Umwelt, Natur and Digitalisierung): leukemic cells were injected intravenously into female NSG-Hc mice (6-10 weeks of age) and leukemic engraftment was followed by detection of human CD45+/murine CD45−/human CD19+ cells in the peripheral blood via flow cytometry analysis. Animals were sacrificed when showing signs of overt leukemia (detection of >75% leukemic blasts in the peripheral blood or clinical signs of leukemia including loss of weight or activity, organomegaly, hindlimb paralysis). Mouse survival was assessed using Kaplan-Meyer log-rank statistics.

In minimal residual disease (MRD) experiments, mice were injected with 10,000 BCP-ALL patient derived xenograft cells (n=10) of E2A-PBX1 positive patients (n=2 patients) and antibody N13B2-HVL6 (5 mg/kg), EFFI-3-VH3VL3 (1 mg/kg) or vehicle were injected intravenously every 3 days starting from day 1 until day 21, when injections were applied every 14 days. Minimal residual disease was measured by PCR for patient-specific immunoglobulin/B-cell receptor rearrangements in bone marrow samples isolated from PDX mice.

In overt leukemia experiments, mice were injected with 1 million BCP-ALL patient derived xenograft cells (n=10) of E2A-PBX1 positive patients (n=2 patients). Once the leukemic engraftment (determined by detection of hCD45+/hCD19+/mCD45− cells in the peripheral blood) was superior to 1%, antibody N13B2-hVL6 (5 mg/kg), EFFI-3-VH3VL3 (1 mg/kg) or vehicle were injected intravenously every 3 days seven times, and subsequently every 14 days.

Antibodies used in treatments. All antibodies were generated at OSE and found free from endotoxin.

Phagocytosis assays. In vitro phagocytosis assays were performed by 1-hour coculture of 2.5×10⁴ human M1 macrophages labeled with CellTrackerGreen (ThermoFisher, Waltham, Mass., USA, 1/2000, 20 min at 37° C.) and 5×10⁴ leukemic cells labeled with CPD (ThermoFisher, 1/2000 10 min at 37° C.) in serum-free RPMI. Phagocytosis was analyzed by a CytoFLEX flow cytometer (Beckman, Brea, Calif., USA) and analysis using FlowJo software (TreeStar, BD Life Sciences Franklin Lakes, N.J., USAs). The phagocytic index was calculated as follows: fold change of percentage of CPD+ cells in CTG+ macrophages compared to the one detected by treatment with istotype control multiplied by the fold change in geometric mean in APC fluorescence (CPD) in CTG+ macrophages compared to the one detected with isotype control. The normalized phagocytic index defines the maximal response by each independent donor against each cell line as 100%, as described in Ring et al. PNAS 2017.

Visualization of engulfed leukemic cells (CPD+) by M1 human macrophages (CTG+) was investigated in parallel to flow cytometry analysis using a Nikon ECLIPSE Ti2 microscope using the NIS-Elements software (Nikon, Minato City, Tokyo, Japan).

Time-lapse microscopy experiments were performed in Ibidi 18-well plates coated with Poly-L-Lysine 0,001%. M1 human macrophages were labeled with pHrodo-SE (ThermoFisher) diluted at 1/333000 for 30 min at 37° C. and seeded at 0.1×106 cells per well. Images were taken every 5 minutes for 4 hours and every 15 minutes for 10 hours by a Nikon ECLIPSE Ti2 microscope using the NIS-Elements software (Nikon).

Phase I study. A First in Human, Phase 1, randomized, double blind, placebo-controlled, single center study (EUDRACT number 2018-001832-22) was conducted in 63 healthy adult male and female volunteers in order to evaluate the safety, tolerability, PK, pharmacodynamics and immunogenicity of single and repeat ascending doses of N13B2-hVL6. N13B2-hVL6 was either administered at single dose (0.002, 0.02, 0.2, 1, 4, or 10 mg/kg IV) or two doses were given 2 weeks apart (6 or 10 mg/kg) and blood samples were drawn in order to evaluate lymphocyte counts after treatment at each time point of the study.

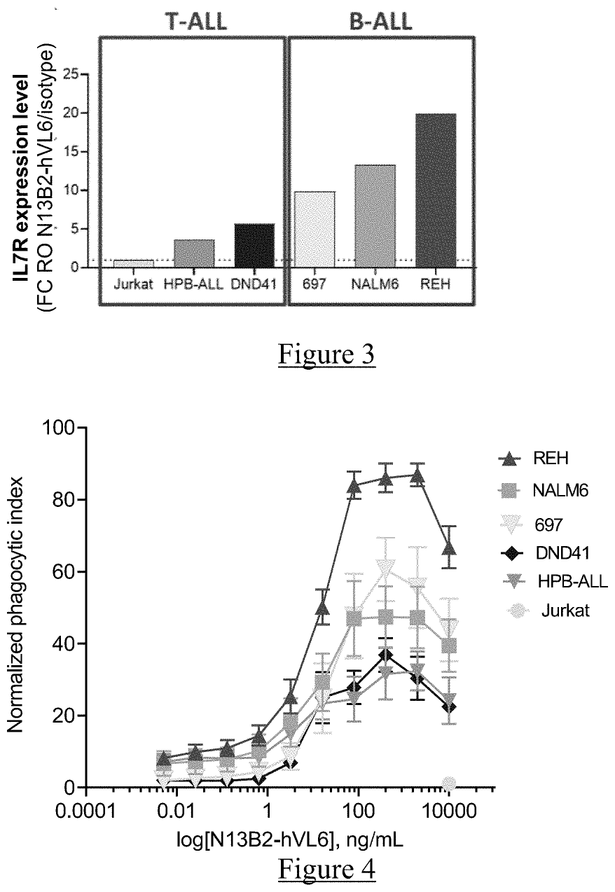

Quantification of specific antibody binding to CD127. N13B2-h L6 and a corresponding isotype control (MOTA IgG4 S228P) were used to label cells (10 ug/mL each, 30 min at 4° C.). A secondary anti-human IgG Fc [HP6017] Mouse IgG2a, κ PE antibody (BioLegend, San Diego, Calif., USA, cat #409304) was used to detect the level of binding of N13B2-hVL6 to the different cell lines. The fold change of receptor occupancy of N13B2-hVL6 (FC RO) in FIG. 3 was calculated as the fold change of Geometric Mean PE fluorescence of N13B2-hVL6 labelled cells compared to that of isotype control labelled cells.

ADCC assay. 1 million human freshly isolated T cells were labelled with 15 uL ⁵¹Cr (5 mCi/ml, PerkinElmer, Waltham, Mass., USA, cat #NEZ030001MC) for 1 h at 37° C., 5% CO2. T cells were then washed until radioactivity (measured by radioactive gamma counter) was absent in the supernatant. 25 μl/well of T cells-⁵¹Cr target cells at 0.4 million cells/mL (10,000 cells/w) were seeded on P96-microtiter plate (flat bottom). 25 μl/well of anti-hCD127 antibodies were added in triplicate at 200 ng/mL (100 ng/mL final concentration) and left to incubate for 15 min at RT. Eventually, 50 μl of NK cells at 2 million cells/mL (100,000 cells/w) (ratio 10 NK cells:1 T cell) were added and incubated for 4 hours at 37° C., 5% CO2. As positive control of cytotoxicity, 75 μL of SDS 10% was added in 3 wells for 10 min before reading. Eventually, 25 μl/w supernatant were placed into radioactive reader plate and 100 μL/w Microscint Scintillant (PerkinElmer cat #60136211) were added. Release of ⁵¹Cr in supernatant was measured by radioactive gamma counter in counts per minutes (cpm). Specific ADCC in FIG. 10 B corresponds to sample well mean cpm (triplicates).

ELISA binding to FcγR. For binding ELISA assay, recombinant hCD64/FcγRI (R&Dsystems, Minneapolis, Minn., USA; reference 1257-FC-050) or hCD32a/FcγRIIa (R&Dsystems, Minneapolis, Minn., USA; reference 1330-CD-050) or hCD16a/FcγRIIIa (R&Dsystems, Minneapolis, Minn., USA; reference 4325-FC-050) was immobilized on plastic at 2 μg/ml in borate buffer (pH9) and purified antibody were added to measure binding. After incubation and washing, peroxidase-labeled donkey anti-human IgG (Jackson Immunoresearch; USA; reference 709-035-149) was added and revealed by conventional methods.

### Results

### Anti-CD127 Agent Effect in Minimal Residual Disease Analysis in a PDX Experiment

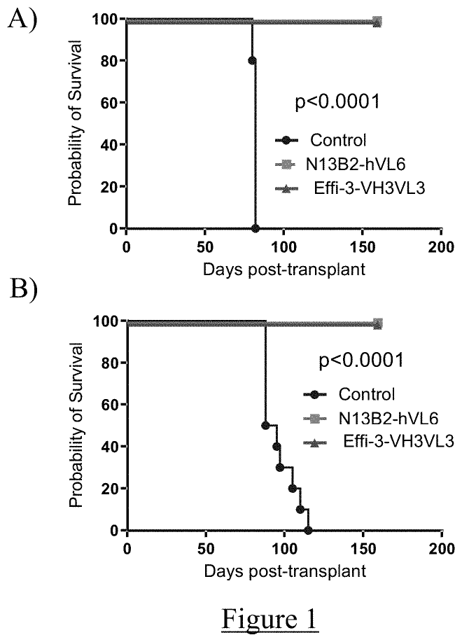

Minimal residual disease (MRD) is the name given to small numbers of leukemic cells that remain in the patient during treatment or after treatment when the patient is in remission (no symptoms or signs of disease). It is the major cause of relapse in leukemia. As illustrated on FIG. 1A, mice with a leukemia patient-derived xenograft (PDX) treated with an anti-CD127 agent having ADCP capabilities over CD127-positive tumor cells all survive during the entire time of the experiment (160 days), while all mice that have been treated with a control were dead after 80 days post-transplantation. Further, it should be noted that 100% of mice were MRD negative, illustrating the anti-leukemic effect of the anti-CD127 agent administrated to the mice, and the potential to fully and definitively treat leukemia. The same result is illustrated on FIG. 1B, wherein mice received a xenograft from another patient. Again, it is shown that mice treated with an anti-CD127 agent having ADCP capabilities towards CD127-positive tumor cells survive, on the contrary to mice treated with a control compound, and further that most of the mice (between 80% and 90% treated with the anti-CD127 agent are MRD negative.

### Effect of Administering an Anti-CD127 Agent in an Overt Leukemia Model

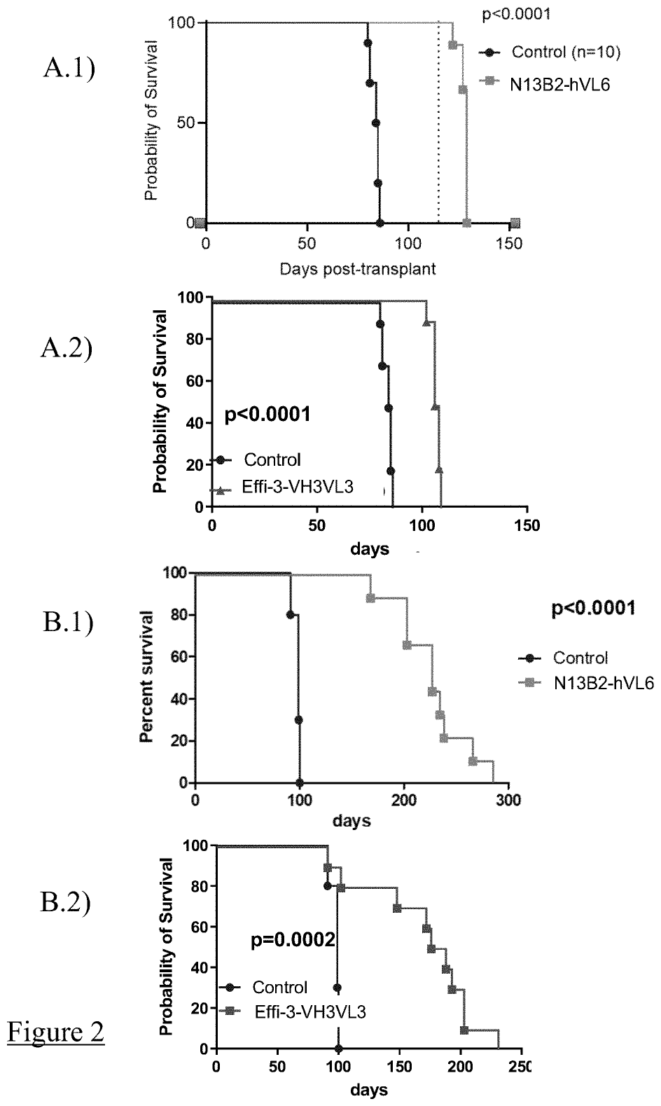

Overt leukemia is the setting where animals are treated when the disease is already well established in the host animal (presence of leukemic blasts over 1 to 5% in the peripheral blood). As illustrated in FIG. 2, PDX mice that have developed an overt leukemia and that were treated with an anti-CD127 agent as defined in the present application survived longer than mice treated with a control compound. In the first xenograft experiment (FIGS. 1A1 and 1A2), the mice treated with the anti-CD127 agent survived between 25% and 50% longer than mice treated with the control antibody. It should be noted that these results were obtained irrespectively of the antagonistic property of the anti-CD127 agent; indeed, even the mice treated with a neutral (i.e. not antagonistic nor agonistic) anti-CD127 agent survived longer than untreated mice. The same results have been obtained in a second experiment (FIGS. 2B1 and 2B2). In this second experiment, it can be seen that some mice treated with anti-CD127 agents that have ADCP capabilities towards CD127-positive tumor cells survived more than 200 days after transplant, twice longer than mice treated with a control. This survival rate is observed irrespectively of the antagonistic capability of the anti-CD127 agent administered to the mice; the same result is observed for mice treated with an antagonistic anti-CD127 agent (FIGS. 2A1 and 2B1) and for mice treated with a neutral (i.e. not antagonist nor agonist) anti-CD127 agent (FIG. 2A2 and 2B2).

### In Vitro Effect on the Phagocytosis of Tumor Cell When an Anti-CD127 Agent is Administered

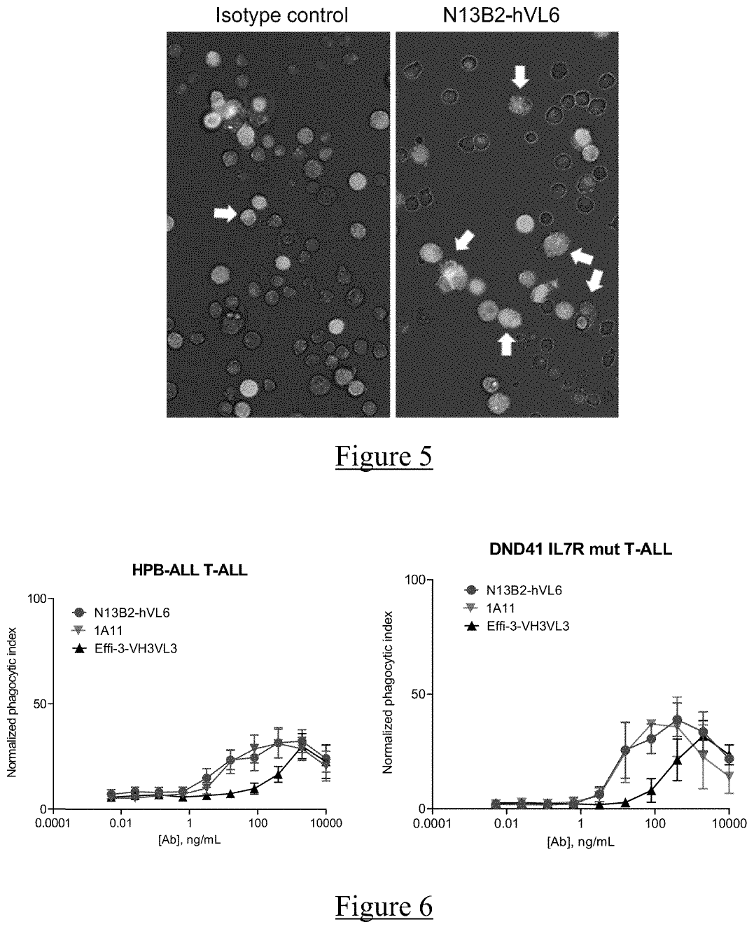

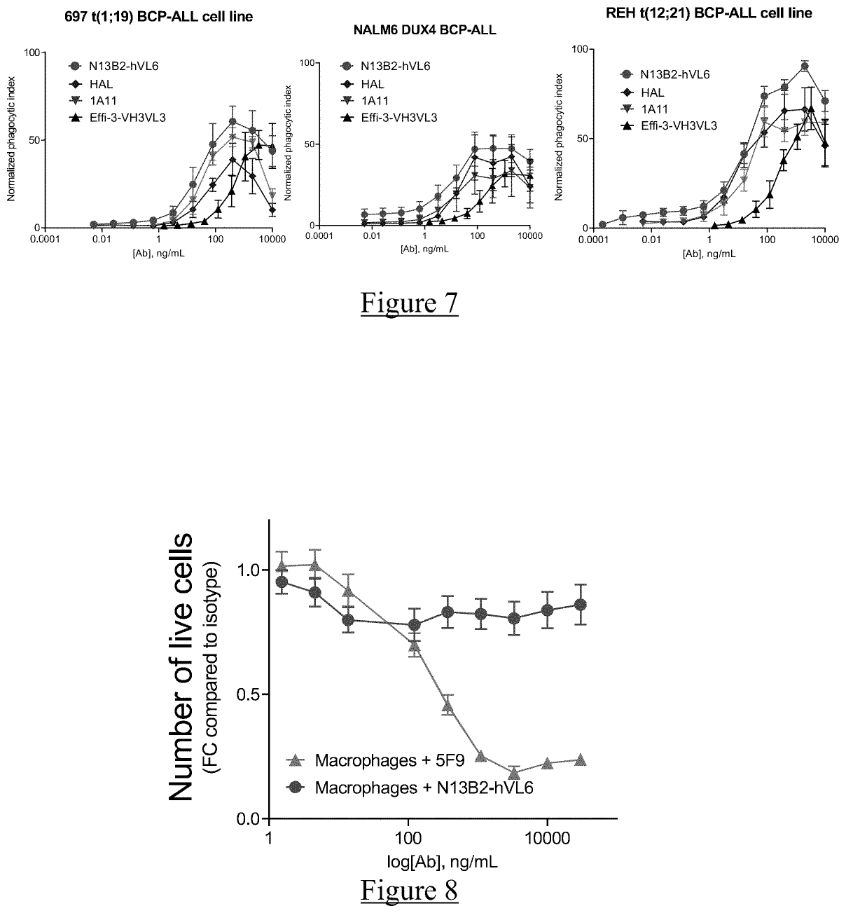

As illustrated on FIG. 3, CD127 expression (assessed by specific N13B2-hVL6 binding to CD127) is variable in different examples of T-cell ALL (HPB-ALL, and CD127 mutated DND41) and B-cell ALL (697, NAML6, and REH) cell lines, or is absent (Jurkat T-ALL cell line), as defined by using an isotype control as a negative control for the evaluation of CD127 expression level. As illustrated on FIG. 4, wherein no cell pre-treatment has been performed, the phagocytosis of CD127 positive tumor cells is enhanced by an anti-CD127 agent, namely N13B2-hVL6 in all ALL lines expressing CD127, the level of phagocytosis achieved associating with the level of CD127 expression (probed by measurement of specific N13B2-hVL6 binding to CD127). As shown on FIG. 5, the administration of N13B2-hVL6 leads to the phagocytosis of leukemia cells by macrophages, illustrating the positive effect of the anti-CD127 agent to induce, sustain, or enhance the phagocytosis of tumor cells. On FIGS. 6 and 7, the capability of several different anti-CD127antibodies to enhance phagocytosis of tumor cells issued from five ALL cell lines expressing CD127 has been tested. Four anti-CD127 antibodies, namely N13B2-hVL6 (with ADCP capability but no ADCC capability: ADCP+/ADCC−), EFFI-3-VH3VL3 (both in-house antibodies), HAL (initially designed by Pfizer and produced in-house) and 1A11 (initially designed by GlaxoSmithKline and produced in-house) all three having ADCP and ADCC capabilities (ADCP+/ADCC+) have been administered at increasing doses on two T-cell ALL cell lines (HPB-ALL and DND41 IL7R mut.) and three B-ALL cell lines (697 t(1;19), NAML6 (DUX4) and REH t(12;21)) in presence of human macrophages. The phagocytosis of tumor cells by macrophages has been assessed according to the method described here above. On FIG. 6, it is shown that all three anti-CD127 antibodies enhance the phagocytosis of T-ALL tumor cells by macrophages. While it may be considered that the anti-CD127 antibody EFFI-3-VH3VL3 is less efficient to enhance the phagocytosis of tumor cells by macrophages, it should be noted that this antibody is less affine for its target CD127 than the other tested antibodies. It can be seen that the anti-CD127 antibody N13B2-hVL6 is very efficient in enhancing the phagocytosis of tumor cells by macrophages. Similar results are illustrated on BALL-cell lines on FIG. 7. The anti-CD127 agents are all able to enhance the phagocytosis of B-ALL tumor cells by macrophages. The anti-CD127 antibody N13B2-hVL6 is the most efficient to enhance the phagocytosis of B-ALL tumor cells.

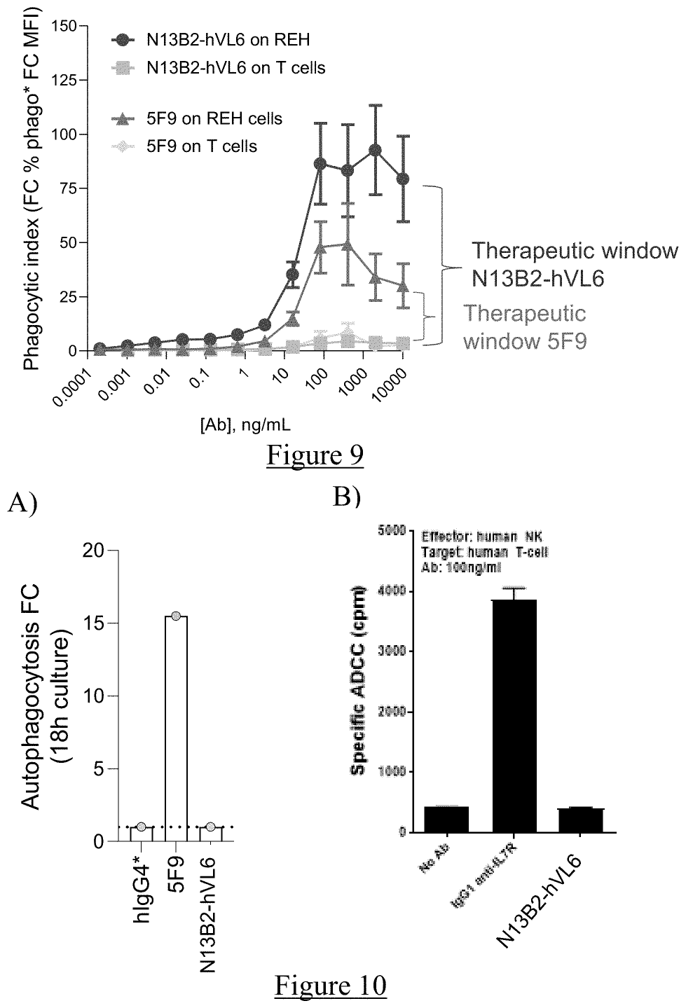

To sum up, these results illustrate that while all anti-CD127 agents tested are efficient to enhance the phagocytosis of CD127-positive tumor cells by macrophages through the ADCP mechanism of action, irrespectively of the type of ALL, including CD127 mutated ALL, N13B2-HVL6 (ADCP+ADCC−) demonstrated the strongest ADCP capability against CD127-positive tumor cells by macrophages, at levels that can surpass that of the reference anti-CD47 antibody 5F9 antibody (see FIG. 9).

### In Vitro Toxicity Effect on Macrophages and Healthy T Cells When an Anti-CD127 Agent of the Invention is Administered, and Lack of ADCC Activity on Human T Cells

The toxicity (i.e. deleterious effect like cell apoptosis or other mechanisms leading to the loss of viable cells) of an anti-CD127 antibody (N13B2-hVL6) or an anti-CD47 antibody (5F9) on macrophages has been assessed and the results are illustrated on FIG. 8. As illustrated, the overall number of live macrophages is not impacted by the dose of anti-CD127 antibody added; it means that the anti-CD127 antibody does not lead to a reduction of the overall number of macrophages, irrespectively of its dosage. On the contrary, when an anti-CD47 agent is administered, the overall number of macrophages is drastically reduced with the administered doses, suggesting that the anti-CD47 agent has a toxic effect on macrophages that leads to their depletion.

According to these results, the ADCP+/ADCC− N13B2-hVL6 antibody does not have a negative impact on the overall population of macrophages, and does not have any adverse effect on their capability to phagocytose tumor cells, unlike other agents currently used in the treatment of ALL.

The phagocytosis of tumor cells (from the REH cell line) and normal T cells by macrophages has been assessed in presence of an ADCP+/ADCC− anti-CD127 antibody (N13B2-hVL6) and an anti-CD47 antibody (5F9), which is a positive control for potent induction of phagocytosis. The results are illustrated on FIG. 9. The anti-CD127 antibody does not have any significant impact on the phagocytosis of normal T cells by macrophages. A similar result is obtained when an anti-CD47 agent is administered. However, the administration of an ADCP+/ADCC− anti-CD127 antibody (N13B2-hVL6) leads to a higher increase in phagocytosis of the tumor cells than that of an anti-CD47 agent. These results mean that the anti-CD127 agents are more likely to enhance the phagocytosis of tumor cells while leaving the normal T cells unharmed at different doses. By combining the results illustrated on FIGS. 8 and 9, the inventors show for the first time that the ADCP+/ADCC− anti-CD127 agents of the invention do not lead to macrophage depletion, do not lead to normal T cell phagocytosis, while they greatly enhance CD127-positive tumor cells phagocytosis by macrophages. These results are further confirmed by the data illustrated on FIG. 10A, which correspond to the phagocytosis of macrophages by macrophages (termed here “autophagocytosis”) in presence of an ADCP+/ADCC− anti-CD127 antibody (N13B2-hVL6) or an anti-CD47 antibody (5F9). In presence of the anti-CD47 antibody, the macrophages have an autophagocytosis activity, due to the expression of CD47 by macrophages. When the anti-CD127 antibody is administered, there is no autophagocytosis of macrophages. These results clearly illustrate once again the lack of toxicity of the anti-CD127 antibody. Further, the ADCC of human T cells by Natural Killer cells induced by the anti-CD127 agent used in the invention has been assessed. The results are illustrated on FIG. 10B. As illustrated, the anti-CD127 agent which has ADCP capability and which does not induce ADCC does not lead to lymphodepletion, contrary to the positive control, which is an antibody that binds to the same target, but which is known for enhancing ADCC activity.

### Lack of In Vivo Toxicity on Healthy Lymphocytes in Humans When an Anti-CD127 Agent is Administered

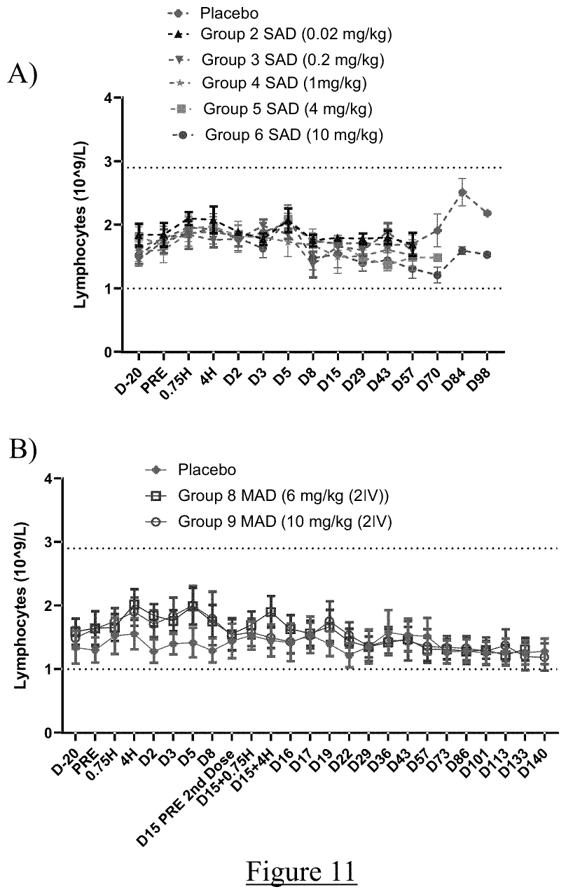

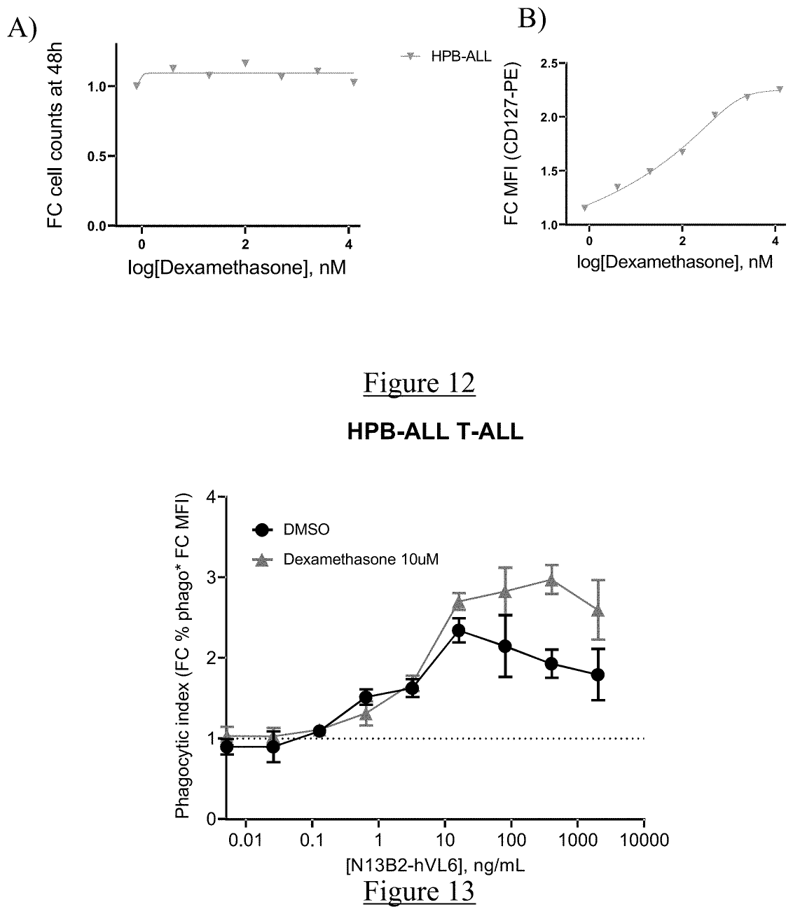

The toxicity (i.e. deleterious effect like cell apoptosis or other mechanisms leading to the loss of viable cells) of an ADCP+/ADCC− anti-CD127 antibody (N13B2-hVL6) on human lymphocytes in vivo has been assessed during a phase 1 clinical trial (EUDRACT number 2018-001832-22) and the results are illustrated on FIG. 11. The administration of single dose of N13B2-hVL6 (0.002, 0.02, 0.2, 1, 4, or 10 mg/kg IV or 1 mg/kg SC) or two doses given 2 weeks apart (6 or 10 mg/kg) was safe and well-tolerated. In all subjects exposed to N13B2-hVL6 up to 10 mg/kg (single and double doses) no clinically significant lymphopenia was reported after N13B2-hVL6 administration.

### Effect of Administering an Anti-CD127 Agent Alone or in Combination in a CD127 Positive ALL Cell Line

As discussed in the description of the invention, several forms of leukemias are resistant to current treatment. As an example, dexamethasone is used to treat different forms of leukemias, but several T-cell ALL and B-cell ALL are known to be resistant to dexamethasone, such as the HPB-ALL cell line (see FIG. 12A) Interestingly, in response to increasing concentrations of dexamethasone treatment, CD127 expression was increased in this cell line in a dose dependent fashion (FIG. 12B).

The ADCP+/ADCC− anti-CD127 antibody N13B2-hVL6 has been administered to the T-ALL HPB-ALL cell line in presence or absence of dexamethasone. As illustrated in FIG. 13, a synergetic effect of the combination of the anti-CD127 N13B2-hVL6 antibody and dexamethasone can be observed in HPB-ALL cells. These results mean that the use of anti-CD127 agents with ADCP+/ADCC− capabilities is efficient to enhance the phagocytosis of tumor cells by macrophages, and may be efficient to treat CD127-positive cancer, but it also means that these ADCP+/ADCC− anti-CD127 agents may be useful to treat patient that have a CD127-positive cancer that is resistant to current therapies, like dexamethasone therapy.

### Effect on the Phagocytosis of Tumor Cells by Macrophage in Presence of Different Anti-CD127 Agents

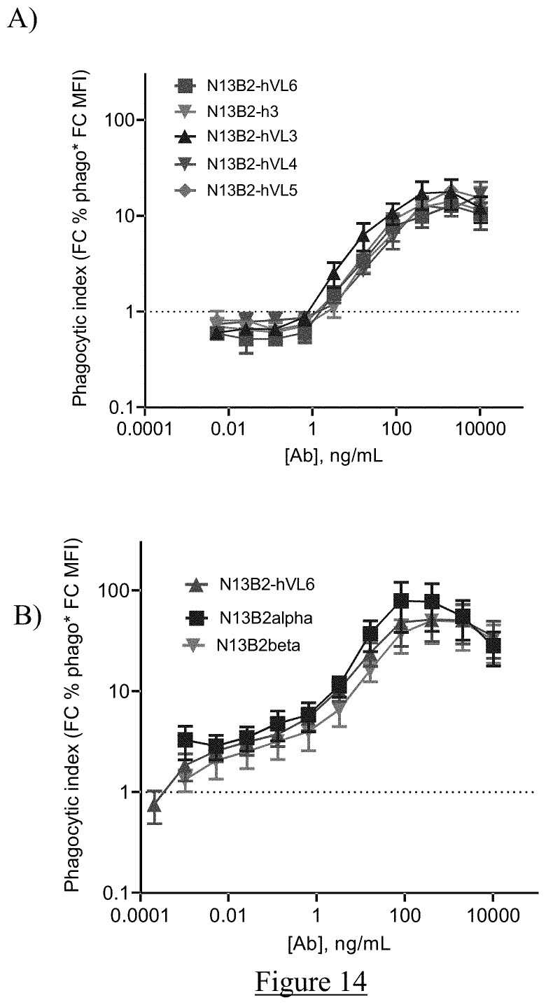

Several anti-CD127 antibodies corresponding to their definition in the description of the invention have been tested to assess their capabilities to enhance the phagocytosis of tumor cells by macrophages. As illustrated on FIG. 14A, anti-CD127 antibodies which share the same CDR domains but different frameworks and on FIG. 14B, anti-CD127 antibodies which share close related CDR domains (with only one or two mutations within the 6 CDRs domains) have all the same capability to enhance the phagocytosis of tumor cells by macrophages.

### Effect on the Binding to FCγR of Different Anti-CD127 Agents

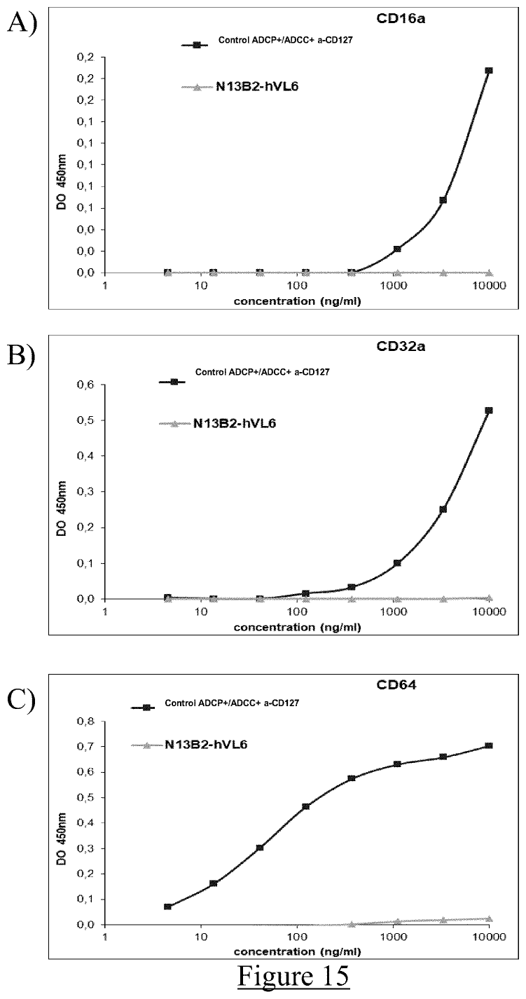

Investigation of N13B2-hVL6 to main activating FCγR by ELISA technology (FIG. 15) indicates that, contrary to a positive control ADCP+/ADCC+ anti-CD127 antibody, N13B2-hVL6 does not bind efficiently to CD16a (FIG. 15A), CD32a (FIG. 15B) or CD64 (FIG. 15C), further highlighting the unexpected capacity of N13B2-hVL6 to induce robust ADCP in CD127 positive tumor cells. These three CD markers bind antibodies through the Fc domain of the antibodies, thereby inducing ADCC and ADCP. As illustrated, the antibody that has an IgG1 domain binds to these three CD markers, which was intended, and may accordingly induce cell clearance mechanisms through ADCC and ADCP. But the anti-CD127 antibodies that are IgG4 do not bind to these markers, which can explain the lack of ADCC capability of these antibodies. Nonetheless, the ADCP capabilities of these antibodies are thus unexpected, because ADCP mechanism is especially mediated by these three CD markers.

1. A method for treating a patient having a CD127-positive cancer by enhancing the phagocytosis of CD127-positive tumor cells, in particular by macrophages, wherein the method comprises the administration to the patient of an effective amount of an anti-CD127 agent, in particular an anti-CD127 antibody or antigen-binding fragment thereof or antigen-binding antibody mimetic, that has Antibody Dependent Cellular Phagocytosis (ADCP) activity on CD127-positive tumor cells, in particular by macrophages cells, and that does not have Antibody Dependent Cytotoxic Activity (ADCC), in particular on immune cells, more particularly on T cells.

2. The method according to claim 1, wherein the CD127-positive cancer is Leukemia, in particular is Acute Lymphoblastic Leukemia (ALL), more particularly is T-cell ALL or B-cell ALL, more preferably is B-cell ALL.

3. The method according to claim 1, wherein the CD127-positive cancer is selected from the group consisting of CD127 overexpressing Acute Lymphoblastic Leukemia (ALL), CD127 and/or JAK-STAT pathway mutated ALL, BCR-ABL1-like ALL, and B cell precursor ALL bearing one the following cytogenetics: t(1;19), t(12,21), MLL-rearrangements, hyperdiploid karyotypes, trisomy 4 and trisomy 10.

4. The method according to claim 1, wherein the CD127-positive cancer is treated by the phagocytosis of CD127-positive tumor cells, in particular by macrophages.

5. The method according to claim 1, wherein the anti-CD127 agent is an anti-CD127 antibody or antigen-binding fragment thereof, comprising a constant chain belonging to the subclass of IgG1, IgG2, IgG3 or IgG4, in particular the subclass of mammalian IgG1, IgG2, IgG3 or IgG4, more particularly the subclass of mammalian IgG4.

6. The method according to claim 1, wherein the anti-CD127 agent is an anti-CD127 antibody or antigen-binding fragment thereof, which comprises:
a VH chain comprising at least the following amino acid sequences:
VHCDR1 SEQ ID No. 3;
VHCDR2 SEQ ID No. 4;
VHCDR3 SEQ ID No. 5 or SEQ ID No. 6;
and a VL chain comprising at least the following amino acid sequences:
VLCDR1 SEQ ID No. 7 or SEQ ID No. 8;
VLCDR2 SEQ ID No. 9 or SEQ ID No. 10;
VLCDR3 SEQ ID No. 11.

7. The method according to claim 1, wherein the anti-CD127 antibody or antigen-binding fragment thereof is an antagonist of the IL7-R signaling pathway induced by the binding of IL7 to CD127.

8. The method according to claim 1, wherein the method further comprises the administration of at least one second therapeutic agent selected from the group consisting of an anti-CD3 agent, in particular anti-CD3 antibody, anti-CD19 agent, in particular an anti-CD19 antibody, and anti-CD47 agent, in particular an anti-CD47 antibody, more particularly an anti-CD47 antagonist agent, even more particularly an anti-CD47 antagonist antibody, an inhibitor of the tyrosine/kinase pathway, Dexamethasone, rituximab, trastuzumab, cetuximab, Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Doxorubicin Hydrochloride; Gleevec (Imatinib Mesylate); Iclusig (Ponatinib Hydrochloride); Inotuzumab Ozogamicin; Imatinib Mesylate; Kymriah (or Tisagenlecleucel); Marqibo (Vincristine Sulfate Liposome); Mercaptopurine (or Purinethol or Purixan); Methotrexate Sodium (or Trexall); Nelarabine; Oncaspar (or Pegaspargase); Ponatinib Hydrochloride; Prednisone; Purinethol (Mercaptopurine); Vincristine Sulfate, Vincristine Sulfate Liposome, and more particularly Dexamethasone.

9. The method according to claim 8, wherein the second therapeutic agent is Dexamethasone.

10. The method according to claim 8, wherein the administration of the anti-CD127 agent and the second therapeutic agent is simultaneous, separate or sequential.

11. A method for treating Acute Lymphoblastic Leukemia (ALL) in a patient by enhancing the Antibody Dependent Cellular Phagocytosis of ALL cells, in particular by macrophages, in particular T-cell ALL or B-cell ALL, more particularly CD127 overexpressing ALL, CD127 and/or JAK-STAT pathway mutated ALL, BCR-ABL1-like ALL, and B cell precursor ALL bearing one the following cytogenetics: t(1;19), t(12,21), MLL-rearrangements, hyperdiploid karyotypes, trisomy 4 and trisomy 10, wherein the method comprises the administration to the patient of an effective amount of an anti-CD127 agent, in particular an anti-CD127 antibody or antigen-binding fragment thereof or antigen-binding antibody mimetic, that has Antibody Dependent Cellular Phagocytosis (ADCP) activity on CD127-positive tumor cells, in particular by macrophages cells, and that does not have Antibody Dependent Cytotoxic Activity (ADCC), in particular on immune cells, more particularly on T cells.

12. The method according to claim 11, wherein the anti-CD127 agent is an anti-CD127 antibody or antigen-binding fragment thereof, which comprises:
a VH chain comprising at least the following amino acid sequences:
VHCDR1 SEQ ID No. 3;
VHCDR2 SEQ ID No. 4;
VHCDR3 SEQ ID No. 5 or SEQ ID No. 6;
and a VL chain comprising at least the following amino acid sequences:
VLCDR1 SEQ ID No. 7 or SEQ ID No. 8;
VLCDR2 SEQ ID No. 9 or SEQ ID No. 10;
VLCDR3 SEQ ID No. 11.

13. The method according to claim 11, wherein the method further comprises the administration at least one second therapeutic agent selected from the group consisting of an anti-CD3 agent, in particular anti-CD3 antibody, anti-CD19 agent, in particular an anti-CD19 antibody, and anti-CD47 agent, in particular an anti-CD47 antibody, more particularly an anti-CD47 antagonist agent, even more particularly an anti-CD47 antagonist antibody, an inhibitor of the tyrosine/kinase pathway, Dexamethasone, rituximab, trastuzumab, cetuximab. Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Doxorubicin Hydrochloride; Gleevec (Imatinib Mesylate); Iclusig (Ponatinib Hydrochloride); Inotuzumab Ozogamicin; Imatinib Mesylate; Kymriah (or Tisagenlecleucel); Marqibo (Vincristine Sulfate Liposome); Mercaptopurine (or Purinethol or Purixan); Methotrexate Sodium (or Trexall); Nelarabine; Oncaspar (or Pegaspargase); Ponatinib Hydrochloride; Prednisone; Purinethol (Mercaptopurine); Vincristine Sulfate, Vincristine Sulfate Liposome, and more particularly Dexamethasone

14. The method according to claim 13, wherein the administration of the anti-CD127 agent and the second therapeutic agent is simultaneous, separate or sequential.

15. The method according to claim 13, wherein the second therapeutic agent is dexamethasone.

16. A method for treating a patient having a CD127-positive cancer, wherein the method comprises the steps of:
a) Determining if the patient has CD127-positive tumor cells,
b) When the patient has a CD127-tumor cells, administrating to the patient an effective amount of an anti-CD127 agent, in particular an anti-CD127 antibody or antigen-binding fragment thereof or antigen-binding antibody mimetic, that has Antibody Dependent Cellular Phagocytosis (ADCP) activity on CD127-positive tumor cells, in particular by macrophages cells, and that does not have Antibody Dependent Cytotoxic Activity (ADCC), in particular on immune cells, more particularly on T cells.

17. The method according to claim 16, wherein the CD127-positive cancer is selected from the group consisting of Acute Lymphoblastic Leukemia (ALL), in particular T-cell ALL or B-cell ALL, more particularly CD127 overexpressing ALL, CD127 and/or JAK-STAT pathway mutated ALL, BCR-ABL1-like ALL, and B cell precursor ALL bearing one the following cytogenetics: t(1;19), t(12,21), MLL-rearrangements, hyperdiploid karyotypes, trisomy 4 and trisomy 10.

18. The method according to claim 16, wherein an effective amount of a second therapeutic agent selected from the group consisting of an anti-CD3 agent, in particular anti-CD3 antibody, anti-CD19 agent, in particular an anti-CD19 antibody, and anti-CD47 agent, in particular an anti-CD47 antibody, more particularly an anti-CD47 antagonist agent, even more particularly an anti-CD47 antagonist antibody, an inhibitor of the tyrosine/kinase pathway, Dexamethasone, rituximab, trastuzumab, cetuximab. Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Doxorubicin Hydrochloride; Gleevec (Imatinib Mesylate); Iclusig (Ponatinib Hydrochloride); Inotuzumab Ozogamicin; Imatinib Mesylate; Kymriah (or Tisagenlecleucel); Marqibo (Vincristine Sulfate Liposome); Mercaptopurine (or Purinethol or Purixan); Methotrexate Sodium (or Trexall); Nelarabine; Oncaspar (or Pegaspargase); Ponatinib Hydrochloride; Prednisone; Purinethol (Mercaptopurine); Vincristine Sulfate, Vincristine Sulfate Liposome, is administrated to the patient.

19. The method according to claim 16, wherein an effective amount of Dexamethasone is administrated to the patient.

20. The method according to claim 19, wherein the administration of the second therapeutic agent is performed simultaneously, separately or sequentially with the administration of the anti-CD127 agent.
